(19) United States
(12) Patent Application Publication
(10) Pub. No.: US 2026/0357336 A1
(43) Pub. Date: Nov. 12, 2026

(54) DROPLET-BASED SINGLE-CELL EPIGENOMIC ASSAY

(71) Applicant: VIRGINIA TECH INTELLECTUAL PROPERTIES, INC., Blacksburg, VA (US)

(72) Inventors: Chang LU, Blacksburg, VA (US); Qiang ZHANG, Christiansburg, VA (US)

(21) Appl. No.: 19/486,448

(22) PCT Filed: May 23, 2024
(86) PCT No.: PCT/US2024/030824

§ 371 Date: Nov. 21, 2025

Related U.S. Application Data
(60) Provisional application No. 63/503,922, filed on May 23, 2023.

(51) Int. Cl. C12Q 1/6869; C12N 1/06; C12Q 1/34; C12Q 1/6806; C12Q 1/686

(52) U.S. Cl. CPC C12Q 1/6869; C12N 1/06; C12Q 1/34; C12Q 1/6806; C12Q 1/686; G01N 2333/916

(57) ABSTRACT

Methods and systems of single-cell bisulfite sequencing are provided. The single-cell bisulfite sequencing techniques of the disclosure involve single-cell DNA (scDNA) droplets, where the scDNA are fragmented within the scDNA droplets prior to bisulfite conversion. The methods and systems further include barcode droplets, and fusing the scDNA droplets with the barcode droplets to form barcoded scDNA droplets for bisulfite conversion. The bisulfite conversion beneficially occurs within the bisulfite droplets to reduce DNA degradation. The single-cell bisulfite sequencing method and systems provide for bisulfite sequenced nucleic acid libraries.

### RELATED APPLICATIONS

This application claims priority to International PCT Application No. PCT/US2024/030824, filed on May 23, 2024, which claims the benefit under 35 U.S.C. § 119(e) to U.S. Provisional Application No. 63/503,922, filed May 23, 2023, and entitled “Droplet-Based Single-Cell Epigenomic Assay”, the entire contents of which are incorporated herein by reference.

### GOVERNMENT SUPPORT CLAUSE

This invention was made with government support under Grant Nos. R21 HG010195 and R01GM143940, awarded by the National Institutes of Health. The government has certain rights in the invention.

### SEQUENCE LISTING XML

The instant application contains a sequence listing, which has been submitted in XML file format by electronic submission and is hereby incorporated by reference in its entirety. The XML file, created on May 23, 2024, is named 94247-4-WOU1_Sequence Listing.xml and is 25,868 bytes in size.

### TECHNICAL FIELD

This disclosure relates to methods of single cell bisulfite sequencing and more particularly to methods of single cell bisulfite sequencing using bisulfite conversion within droplets.

### BACKGROUND

Genome-wide DNA methylation profile, or DNA methylome, is a critical component of the overall epigenomic landscape that modulates gene activities and cell fate. Single-cell DNA methylomic studies offer unprecedented resolution for detecting and profiling cell subsets based on methylomic features. For example, DNA methylation, such as methylation of cytosine at its C5 position, is an important epigenetic mechanism and plays a critical role in regulating genome-wide expression and activity.

Diverse DNA methylation profiles are associated with various cell types, developmental stages, and disease states. Single-cell whole genome bisulfite sequencing (scWGBS) is a powerful assay to decipher the heterogeneity of cell-type-specific methylomes at the tissue level and genome-wide scale with single-nucleotide resolution. Several tools have been developed to prepare scWGBS libraries in tubes or well plates, including scBS-seq, snmC-seq, snmC-seq2, sciMET, and sciMETv2. In the early scBS-seq technologies, single cells were isolated into tubes and processed individually without involving barcoding, thus, only a small number of cells could be studied. snmC-seq and snmC-seq2 further refined the process toward analyzing a large number of cells by conducting bisulfite conversion in well plates (96 or 384) and using indexed random primers that allowed labeling and pooling of single cells. Furthermore, sciMET was developed based on the use of combinatorial indexing to label single cells or nuclei with unique indexes without physical isolation, while the nuclear integrity was maintained. Although a relatively large number of cells (thousands) could be processed using these technologies by running multiple processes in parallel or for an extended period, these platforms do not intrinsically offer high-speed operation.

A central mission of single-cell analysis is to reveal heterogeneity and identify subpopulations in tissue samples. Therefore, the capacity for handling at least 10⁴-10⁵ single cells is crucial for such tasks. For example, a complete analysis of one core biopsy tumor sample requires screening of about 10⁴ single cells. A tissue sample from a specific region of a postmortem human brain is typically 0.5-1 g in mass, containing roughly 3-6 million neurons and as many glial cells. Therefore, in this case, survey of about 10⁴-10⁵ single cells allows for the sampling of a small, but meaningful, fraction of the total cell population. However, existing single cell methylomic technologies are based on use of tubes or well plates and these platforms are not easily scalable for handling such large number of single cells.

Therefore, there remains a need for single cell methylomic processes that are able to profile various cell types and process higher numbers of single cells.

### SUMMARY

The methods and systems provided herein are suitable for single cell bisulfite sequencing, including methods of single cell bisulfite sequencing using bisulfite conversion within droplets.

In Example 1, a method of single-cell bisulfite sequencing comprises generating a plurality of single-cell DNA (scDNA) droplets comprising a single cell or nucleus from a population of cells or nuclei and a lysis buffer comprising an enzyme; lysing the single cell or nucleus within each of the plurality of scDNA droplets resulting in a plurality of fragmented scDNA within each of the plurality of scDNA droplets; generating a plurality of barcode droplets each comprising a barcode bead oligonucleotide; fusing the plurality of scDNA droplets and the plurality of barcode droplets to form a plurality of barcoded scDNA droplets each comprising a barcoded scDNA; breaking the plurality of barcoded scDNA droplets; co-encapsulating the barcoded scDNA with a bisulfite reagent to form a plurality of bisulfite droplets; converting the barcoded scDNA to bisulfite-converted scDNA within the plurality of bisulfite droplets; and determining methylation profile of the fragmented scDNA.

Example 2 relates to the method according to Example 1, wherein the fragmented scDNA has a size ranging from 100 bp to about 1000 bp prior to the step of converting the barcoded scDNA to the bisulfite-converted scDNA.

Example 3 relates to the method according to Example 1 or 2, wherein the enzyme comprises micrococcal nuclease.

Example 4 relates to the method according to any one of Examples 1-3, wherein the plurality of bisulfite droplets each have a diameter of about 20 μm to about 200 μm.

Example 5 relates to the method according to any one of Examples 1-4, wherein the lysis buffer further comprises CaCl₂) in a concentration range of about 0.1 mM to about 0.3 mM. Example 6 relates to the method according to any one of Examples 1-5, wherein the generation step of the plurality of scDNA droplets occurs before and/or after the generation step of the plurality of barcode droplets.

Example 7 relates to the method according to any one of Examples 1-6, wherein the plurality of scDNA droplets are generated at a rate in the range of about 1000 droplets per second to about 4000 droplets per second, and wherein the plurality of scDNA droplets each have a diameter in the range of about 15 μm to about 50 μm.

Example 8 relates to the method according to any one of Examples 1-7, wherein each of the barcode bead oligonucleotides comprise the following in order from (i) to (iii): (i) a bead for carrying barcode oligonucleotides; (ii) at least one photocleavable linker; and (iii) at least one barcode oligonucleotide sequence comprising: a PCR priming site comprising the sequence CAAGCAGAAGACGGCATACGAGAT (SEQ ID NO: 2); a cell barcode sequence having a list of 10-20 nucleotides comprising A, G, T, or a combination thereof; and a ligation site sequence having a list of 10-50 nucleotides comprising A, G, C, T, or a combination thereof.

Example 9 relates to the method according to Example 8, wherein a complementary oligonucleotide comprising 19 nucleotides is annealed to the ligation site to form a double-stranded tail at a 3-end of the barcode bead oligonucleotide for ligating to a single fragmented scDNA from the plurality of fragmented scDNA to form the barcoded scDNA within the plurality of barcoded scDNA droplets.

Example 10 relates to the method according to Example 8, wherein the plurality of barcoded scDNA droplets are exposed to ultraviolet (UV) radiation to remove the bead from each of the at least one barcode oligonucleotide sequence via breakage of each of the at least one photocleavable linker.

Example 11 relates to the method according to any one of Examples 1-10, wherein the bisulfite reagent comprises a bisulfite salt selected from the group consisting of sodium bisulfite and ammonium bisulfite.

Example 12 relates to the method according to any one of Examples 1-11, wherein the bisulfite-converted scDNA further undergoes one or more steps comprising random priming and/or indexing PCR for generating a bisulfite sequenced nucleic acid library.

Example 13 relates to the method according to Example 12, wherein the indexing PCR comprises indexing-PCR-based amplification to generate i5- and/or i7-indexed libraries.

Example 14 relates to the method according to any one of Examples 1-13, wherein the method produces a bisulfite sequenced nucleic acid library of about 1000 to about 10,000 single cells within a period of about 2 days, and provides a bisulfite conversion rate of at least 95%.

Example 15 relates to the method according to any one of Examples 1-14, wherein the converting of the barcoded scDNA to the bisulfite-converted scDNA within the plurality of bisulfite droplets reduces DNA degradation.

Example 16 relates to the method according to any one of Examples 1-15, wherein the plurality of scDNA droplets, the plurality of barcode droplets, the plurality of barcoded scDNA droplets, and the plurality of bisulfite droplets are water-in-oil droplets.

In Example 17, a system for single-cell sequencing comprises a droplet generation device for generating a plurality of scDNA droplets, the plurality of scDNA droplets comprising a single cell or nucleus from a population of cells or nuclei and a lysis buffer comprising an enzyme; a barcode droplet generation unit within a droplet fusion device, wherein the barcode generation unit generates a plurality of barcode droplets, each comprising a barcode bead oligonucleotide; the droplet fusion device for fusing the plurality of scDNA droplets and the plurality of barcode droplets to form a plurality of barcoded scDNA droplets each comprising a barcoded scDNA; and a bisulfite droplet device for co-encapsulating the barcoded scDNA with a bisulfite reagent to form a plurality of bisulfite droplets.

Example 18 relates to the system according to Example 17, wherein the droplet generation device comprises three separate inlets each attached to a pump to separately administer the single cell or nucleus from the population of cells or nuclei, the lysis buffer, and an oil to form the plurality of scDNA droplets.

Example 19 relates to the system according to Example 17 or 18, wherein the plurality of scDNA droplets are received in a scDNA droplets inlet within the droplet fusion device prior to forming the plurality of barcoded scDNA droplets.

Example 20 relates to the system according to any one of Examples 17-19, wherein the droplet fusion device further comprises one or more electrode channels comprising a salt solution for conducting dielectrophoresis-based droplet fusion to fuse the plurality of scDNA droplets and the plurality of barcode droplets to form the plurality of barcoded scDNA droplets.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF DRAWINGS

FIG. 1 shows a schematic diagram of the droplet bisulfite sequencing technology (Drop-BS) library preparation process. Single cells or nuclei are encapsulated into droplets (scDNA droplets) for lysis and DNA fragmentation. The schematic shows where after merging scDNA droplets with barcode droplets, fragmented DNA is tagged with barcoded oligos released from barcode beads. Barcoded scDNA is then pooled and further co-encapsulated with bisulfite salt into droplets for bisulfite conversion. Another priming site is added to the DNA using random priming reaction in a tube. Full P5/P7 adapters on the DNA fragments are added by PCR.

FIG. 2A shows exemplary method steps including the oligonucleotide and primer sequences involved in the construction of the Drop-BS library.

FIG. 2B continues from FIG. 2A and shows exemplary method steps including the oligonucleotide and primer sequences involved in the construction of the Drop-BS library.

FIG. 3A shows a microscopic image of a droplet generation device.

FIG. 3B shows a microscopic image of a droplet fusion device.

FIG. 3C shows a microscopic image of the device in operation for single-nuclei droplet generation.

FIG. 3D shows a microscopic image of the device in operation for barcode droplet generation in the droplet fusion device.

FIG. 3E shows a microscopic image of the device in operation for scDNA droplet reinjection and pairing with barcode droplets.

FIG. 3F shows a microscopic image of the device in operation for the fusion of scDNA droplets and barcode droplets under dielectrophoresis. The scale bar is 100 μm.

FIG. 4 shows a diagram of the bisulfite droplet device used in Drop-BS. Filtration structures are placed between the inlets and the narrow channels to trap particles and prevent clogging.

FIG. 5 shows a graph of the size profile of a Drop-BS sequencing library measured by an Agilent TapeStation.

FIG. 6 shows a graph of the relationship between the total number of reads and the number of unique reads for each cell in Drop-BS datasets.

FIG. 7A shows a graph relating to the selection of cell-associated barcodes under a mapping efficiency cutoff with human/mouse mixed cell sample. GM12878 and mouse brain nuclei were mixed at 1:1 ratio and Drop-BS library of ~1,000 cells were prepared. Each dot is a barcode bearing reads that can align to human genome or mouse genome. “Pure” barcodes refer to the ones with 90% or more of their reads aligned to one genome (human hg19 or mouse mm10), p and a are the mean and standard deviation of the fitted normal distribution on the right, respectively. In FIG. 7A, μ-0.5α was used as a cutoff in the probability density plot shown (left) and the corresponding alignment of selected barcodes to the human and mouse genomes shown (right). μ-α is selected as the cutoff that balances the data quality (purity) and the number of cells covered.

FIG. 7B shows the data where μ-α was used as a cutoff in the probability density plot shown (left) and the corresponding alignment of selected barcodes to the human and mouse genomes shown (right).

FIG. 7C shows the data where μ-2α was used as a cutoff in the probability density plot shown (left) and the corresponding alignment of selected barcodes to the human and mouse genomes shown (right).

FIG. 7D shows the data where μ-3α was used as a cutoff in the probability density plot shown (left) and the corresponding alignment of selected barcodes to the human and mouse genomes shown (right).

FIG. 8A shows graphs of the probability density plots of mapping efficiency for all samples and their corresponding cut offs (μ-α) for selection of cell-associated barcodes. The graphs show the data for Mixed human and mouse cells, and GM12878. The dot-and-line lines: density distributions of mapping efficiency of Drop-BS data. Solid lines: fitted normal distributions. Dotted lines: combined fitted distributions. Vertical broken lines: the mapping efficiency cutoff, i.e. μ-α of the normal distribution on the right.

FIG. 8B shows graphs of the probability density plots of mapping efficiency for all samples and their corresponding cut offs (μ-α) for selection of cell-associated barcodes. The graphs show the data for HEK293 cells, and MCF7. The dot-and-line lines: density distributions of mapping efficiency of Drop-BS data. Solid lines: fitted normal distributions. Dotted lines: combined fitted distributions. Vertical broken lines: the mapping efficiency cutoff, i.e. μ-α of the normal distribution on the right.

FIG. 8C shows graphs of the probability density plots of mapping efficiency for all samples and their corresponding cut offs (μ-α) for selection of cell-associated barcodes. The graphs show the data for mixed cell lines (GM112878+HEK293+MCF7), and Mouse brain. The dot-and-line lines: density distributions of mapping efficiency of Drop-BS data. Solid lines: fitted normal distributions. Dotted lines: combined fitted distributions. Vertical broken lines: the mapping efficiency cutoff, i.e. μ-α of the normal distribution on the right.

FIG. 8D shows graphs of the probability density plots of mapping efficiency for all samples and their corresponding cut offs (μ-α) for selection of cell-associated barcodes. The graphs show the data for Human brain 1 and Human brain 2 samples. The dot-and-line lines: density distributions of mapping efficiency of Drop-BS data. Solid lines: fitted normal distributions. Dotted lines: combined fitted distributions. Vertical broken lines: the mapping efficiency cutoff, i.e. μ-α of the normal distribution on the right.

FIG. 9A shows a graph of the Drop-BS data on cell lines and their mixture (containing equal portions of the three cell lines). FIG. 9A shows the UMAP visualization of Louvain clustering results on Drop-BS data of a mixture of three human cell lines (Cluster 1=MCF7; 2=HEK293; and 3=GM12878).

FIG. 9B shows a graph of the CG methylation rate (mCG/CG), CH methylation rate (mCH/CH), and mapping efficiency of selected high-quality single-cell data from the mixed sample.

FIG. 9C shows a Pearson correlation and hierarchical clustering among the merged Drop-BS data and published methylomic data based on mCG/CG across equally spaced 1 M genomic bins. “GM12878”, “HEK293”, and “MCF7” were the merged single-cell Drop-BS data when each cell line was individually profiled. “GM12878_mixed”, “HEK293_mixed”, “MCF7_mixed” were the merged single-cell Drop-BS data generated on each cell line by clustering the mixed sample. “GM12878_published”, “HEK293_published” and “MCF7_published” were published bulk methylomic data from ENCODE (ENCFF570TIL), GSM1254259, and GSM1328112, respectively.

FIG. 10A shows a graph of Drop-BS for identifying various cell types in the mixed sample containing three cell lines. In FIG. 10A, the graph includes the co-clustering of Drop-BS data of the mixed sample (1929 cells) together with 100 single-cell data from each cell line.

FIG. 10B shows the co-clustering results of the added single-cell data with known identities and the mixed sample yielding three clusters.

FIG. 10C shows the co-clustering results for mCG/CG for the single-cell data.

FIG. 10D shows the co-clustering of the number of unique reads for the single-cell data.

FIG. 11A shows violin plots for the Drop-BS data on mouse (n=1123 cells) prefrontal cortex tissue. SnmC-seq profiled mouse (n=3377 cells) frontal cortex neurons. sci-MET profiled mouse cortex tissue (n=210 cells). In the violin plots, the lower and upper hinges correspond to the first and third quartiles. The upper whisker extends from the hinge to the largest value no further than 1.5*IQR from the hinge. The lower whisker extends from the hinge to the smallest value at most 1.5*IQR of the hinge. Data beyond the end of the whiskers are plotted individually.

FIG. 11B shows violin plots for the Drop-BS data on human (n=1556 cells for human1 and n=1257 cells for human2) prefrontal cortex tissue. SnmC-seq profiled human (n=2740 cells) frontal cortex neurons. In the violin plots, the lower and upper hinges correspond to the first and third quartiles. The upper whisker extends from the hinge to the largest value no further than 1.5*IQR from the hinge. The lower whisker extends from the hinge to the smallest value at most 1.5*IQR of the hinge. Data beyond the end of the whiskers are plotted individually.

FIG. 12A shows a graph of the methylation level (mCG/CG) across upstream 2 kb of TSS (transcription start site), NCBI RefSeqGene body, and downstream 2 kb of TES (transcription termination site) for single cells (grey lines) and an average of all cells (single solid line) for the mouse PFC sample.

FIG. 12B shows a graph of the methylation level (mCG/CG) across upstream 2 kb of TSS (transcription start site), NCBI RefSeqGene body, and downstream 2 kb of TES (transcription termination site) for single cells (grey lines) and an average of all cells (single solid line) for the Human brain 1 PFC sample.

FIG. 12C shows a graph of the methylation level (mCG/CG) across upstream 2 kb of TSS (transcription start site), NCBI RefSeqGene body, and downstream 2 kb of TES (transcription termination site) for single cells (grey lines) and an average of all cells (single solid line) for the Human brain 2 PFC sample.

FIG. 13A shows a graph of the Drop-BS for differentiating cell types based on single-cell CH methylation in mouse brain tissue. The graph shows a Louvain clustering of Drop-BS data on mouse prefrontal cortex.

FIG. 13B shows CG methylation z-scores of each mouse PFC cluster over known mouse neuron type-specific CG-DMRs. As shown in FIG. 13B, In=inhibitory neurons; Nn=non-neuronal cells; and Ex=excitatory neurons.

FIG. 14A shows a graph of Drop-BS for differentiating cell types based on single-cell CH methylation in human brain tissues. FIG. 14A shows the Louvain clustering of Drop-BS data on human brain 1 (HB1).

FIG. 14B shows a graph of Drop-BS for differentiating cell types based on single-cell CH methylation in human brain tissues. FIG. 14B shows the Louvain clustering of Drop-BS data on human brain 2 (HB2) prefrontal cortex samples.

FIG. 14C shows a graph of Drop-BS for differentiating cell types based on single-cell CH methylation in human brain tissues. FIG. 14C shows the Louvain clustering of Drop-BS data on HB1 and HB2.

FIG. 14D shows a graph of Drop-BS for differentiating cell types based on single-cell CH methylation in human brain tissues. FIG. 14C shows the Louvain clustering of Drop-BS data on HB1 and HB2.

FIG. 14E shows the data of a comparison of clusters generated on single human brain samples with clusters generated on the combined human brain data.

FIG. 14F shows the data of CG methylation z-scores of each of HB1+2 cluster over human neuron type-specific CG-DMRs. In=inhibitory neurons; Ex=excitatory neurons; Nn=non-neuronal cells.

FIG. 14G shows graphs of human marker genes that differentiate inhibitory and excitatory neurons. The single cells are shaded according to their normalized mCH level in the gene body.

FIG. 15A shows a graph of the effect of CaCl₂ concentration on the size distribution of single-cell genomic DNA fragmented in droplets. MNase had a concentration of 0.01875 U/μl in droplets. FIG. 15A shows the results for a CaCl₂ concentration of 0.25 mM. The final sequencing libraries yielded under these conditions (having a volume of 20 μl) had a concentration of 0.5 nM, measured by qPCR using the KAPA library quantification kit.

FIG. 15B shows a graph of the effect of CaCl₂ concentration on the size distribution of single-cell genomic DNA fragmented in droplets. MNase had a concentration of 0.01875 U/μl in droplets. FIG. 15B shows the results for a CaCl₂ concentration of 0.1625 mM. The final sequencing libraries yielded under these conditions (having a volume of 20 μl) had a concentration of 2.5 nM, measured by qPCR using the KAPA library quantification kit.

FIG. 15C shows a graph of the effect of CaCl₂ concentration on the size distribution of single-cell genomic DNA fragmented in droplets. MNase had a concentration of 0.01875 U/μl in droplets. FIG. 15C shows the results for a CaCl₂ concentration of 0.0625 mM. The final sequencing libraries yielded under these conditions (having a volume of 20 μl) had a concentration of 0.1 nM, measured by qPCR using the KAPA library quantification kit.

FIG. 16 shows a graph of the amount of oligonucleotide released from about 5,000 barcode beads after various UV exposure times. All experiments were conducted in triplicate, and the horizontal lines represent the mean.

FIG. 17 shows a graph of the library concentration after bisulfite conversion in droplets and bulk (in a tube). Drop-BS protocol was used to construct these libraries (each starting with about 1,000 GM12878 single cells), except that the “bulk” ones had bisulfite conversion in a tube instead of in droplets. The library concentration was measured using a KAPA Library Quantification Kit. All experiments were conducted in duplicate, and the horizontal lines represent the mean.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The embodiments of this disclosure are not limited to particular methods, which can vary and are understood by skilled artisans. It is further to be understood that all terminology used herein is for the purpose of describing particular embodiments only and is not intended to be limiting in any manner or scope. So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1%, and 4% This applies regardless of the breadth of the range.

The term “about,” as used herein, refers to variation in the numerical quantity that can occur, for example, through typical measuring techniques and equipment, with respect to any quantifiable variable, including, but not limited to, mass, volume, temperature, and time. Further, given solid and liquid handling procedures used in the real world, there is certain inadvertent error and variation that is likely through differences in the manufacture, source, or purity of the ingredients used to make the compositions or carry out the methods and the like. Whether or not modified by the term “about,” the claims include equivalents to the quantities.

The term “amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 2-100 “cycles” of denaturation and replication.

The terms “Polymerase chain reaction,” or “PCR” as used herein, means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. In some cases, the annealing and extension steps may be combined into a single step. Particular temperatures, durations at each step, and rates of change between steps depend on many factors well-known to those of ordinary skill in the art.

The term “primer,” as used herein, means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually, primers are extended by a DNA polymerase. Primers are generally of a length compatible with its use in synthesis of primer extension products, and are usually are in the range of between 6 to 100 nucleotides in length, such as 6 to 70, 10 to 50, 10 to 75, 15 to 60, 15 to 40, 15 to 45, 18 to 30, 18 to 40, 20 to 30, 20 to 40, 21 to 25, 21 to 50, 22 to 45, 25 to 40, and any length between the stated ranges. In some embodiments, the primers are usually not more than about 6, 7, 8, 9, 10, 12, 15, 20, 21, 22, 23, 24, 25, 26, 27, 28, 29, 30, 35, 40, 45, 50, 55, 60, 65, or 70 nucleotides in length.

The term “nucleic acid” or “polynucleotide” as used herein, will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adapter” may also be used interchangeably with the terms “oligonucleotide” and “polynucleotide.” In addition, the term “adapter” can indicate a linear adapter (either single stranded or double stranded) or a stem-loop adapter. These definitions generally refer to at least one single-stranded molecule, but in specific embodiments will also encompass at least one additional strand that is partially, substantially, or fully complementary/to at least one single-stranded molecule. Thus, a nucleic acid may encompass at least one double-stranded molecule or at least one triple-stranded molecule that comprises one or more complementary strand(s) or “complements” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about. 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent, conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least, one single or double-stranded nucleic acid molecule during hybridization.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

Provided herein are methods and systems of single-cell bisulfite sequencing. In some examples, the disclosure includes the use of droplet-based technology to construct single-cell bisulfite sequencing libraries for DNA methylome profiling (a process that may also be referred to herein as “Drop-BS”). In some aspects, Drop-BS takes advantage of the ultrahigh throughput offered by droplet microfluidics to prepare bisulfite sequencing libraries of up to 10,000 single cells within 2 days. Beneficially, the methods as described herein can be used for single-cell methylomic studies or genomic profiling requiring examination of a large cell population.

In aspects, the methods of the disclosure may utilize droplet-based technology. In examples, droplet-based microfluidics can allow for the rapid production and manipulation of millions of water-in-oil droplets as microreactors for the isolation and treatment of single cells. Droplet-based technology may also be successfully used to produce single-cell sequencing libraries with high throughput for RNA-seq, DNA-seq, ATAC-seq, ChIP-seq, CUT&Tag, as well as multi-omics-seq. In these assays, droplet operations, such as encapsulation and fusion, have been implemented for physical isolation of single cells or beads and addition of reagents, respectively. Some of the assays may also involve molecular biology reactions at elevated temperatures within droplets.

As described herein, the methods and systems are directed to high-throughput droplet-based platforms to construct single-cell bisulfite sequencing libraries for DNA methylome profiling. The current challenges for bisulfite sequencing library preparation in droplets lie in genomic DNA fragmentation and bisulfite conversion. For example, DNA fragmentation in bisulfite sequencing has conventionally been done by mechanical DNA shearing, which is not compatible with droplet-based assays. In recent single-cell assays, DNA fragmentation has been done during bisulfite treatment, however, the fragmentation could not be separately optimized independent of the bisulfite conversion process. As described within the present disclosure, the methods and systems provided herein are able to overcome the deficiencies in the art. In some aspects, the methods and systems utilize enzymes to randomly cut genomic DNA into fragments and optimized independently of (i.e., before) the bisulfite conversion process, increasing DNA recovery. Further, conventional bisulfite conversion in tubes currently results in significant DNA degradation. In some aspects, the methods and systems of the disclosure overcome this deficiency by completing bisulfite conversion within droplets, substantially improving DNA recovery. By conducting pre-bisulfite adapter/barcode tagging, the complexity of conducting post-bisulfite adapter/barcode tagging in droplets can be avoided. In some aspects, the methods and systems may be compatible with further droplet-based single-cell technologies.

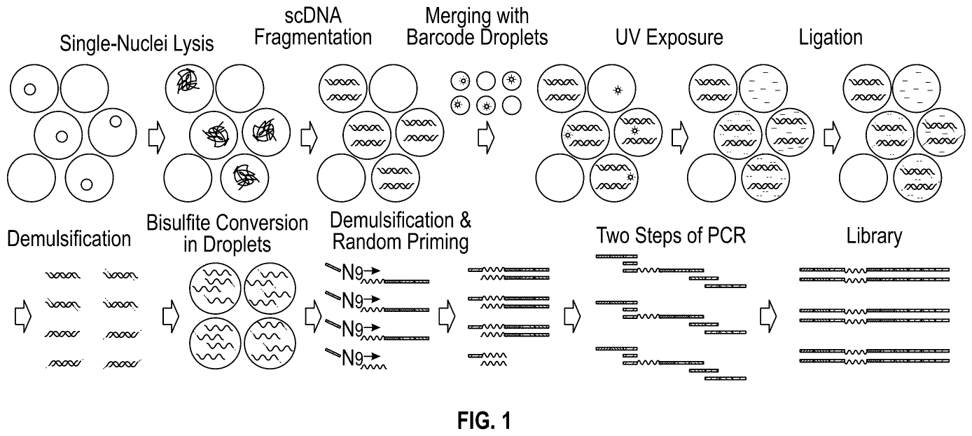

The methods and systems of the disclosure take advantage of the versatility offered by droplet-based techniques for pairing single cells and barcode beads, mixing various reagents, and conducting multi-step reactions. In some aspects, by manipulating single cells and single barcode beads within the droplets, DNA from a single cell can be tagged by a unique DNA barcode that can be further released from a barcode bead. The barcoded DNA may then undergo bisulfite conversion within the droplets. In embodiments, the converted DNA may then undergo random priming and indexing PCR to generate a sequencing library. A brief summary of the methods provided herein may be found in FIG. 1. As shown in FIG. 1, single cells or nuclei are encapsulated into single-cell DNA (scDNA) droplets for lysis and DNA fragmentation. After merging scDNA droplets with barcode droplets, fragmented DNA is tagged with barcoded oligonucleotides released from barcode beads. The barcoded scDNA are then pooled and further co-encapsulated with bisulfite salt into droplets for bisulfite conversion. Another priming site is added to the DNA using random priming reaction in a tube. The full P5/P7 adapters on the DNA fragments are then added by PCR.

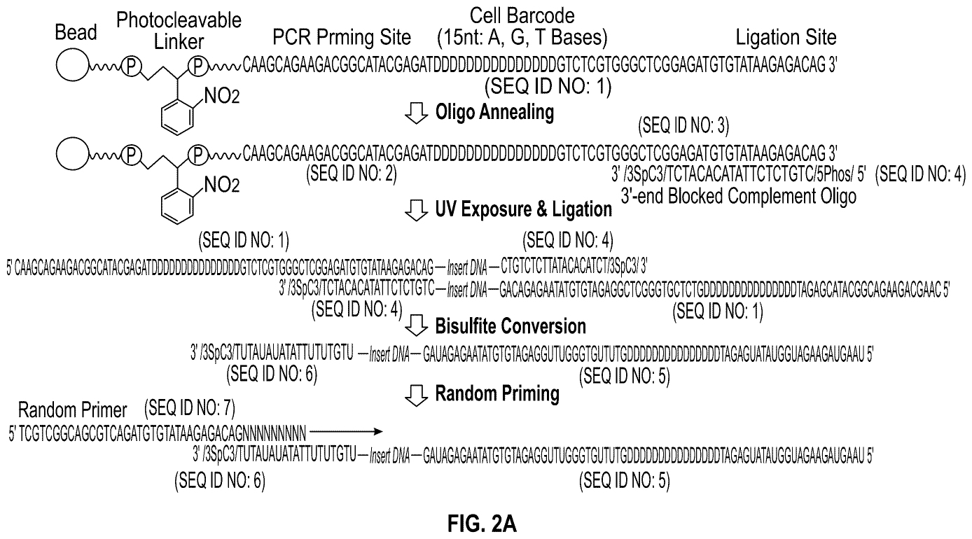

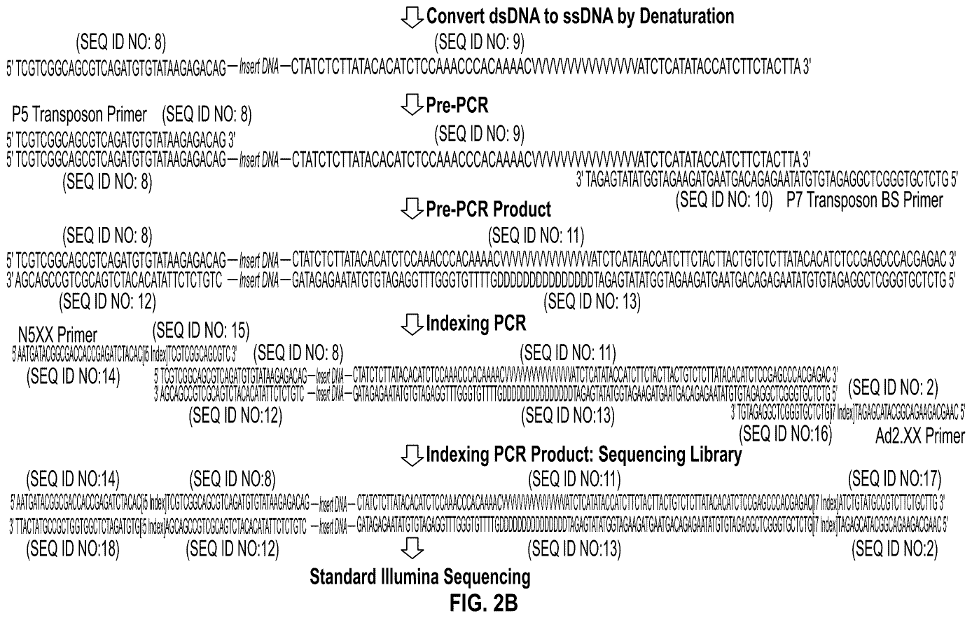

In further embodiments, example oligonucleotide and primer sequences of the disclosure involved in the construction of the sequencing library may be found in FIG. 2A and FIG. 2B. Further details of the steps within the methods and systems of the disclosure are provided herein.

In some aspects, the method of the disclosure comprises a method of single-cell bisulfite sequencing. In embodiments, the method comprises a step of generating a plurality of scDNA droplets. The scDNA droplets may comprise a single cell or nucleus from a population of cell or nuclei, respectively. The cells or nuclei may arise from a human, or from a non-human animal, for example, art invertebrate cell (e.g., a cell from a fruit fly), a fish cell (e.g., a zebrafish cell), an amphibian cell (e.g., a frog cell), a reptile cell, a bird cell, or a mammal cell. If the cell or nuclei is from a multicellular organism, the cell may be from any part of the organism. In some embodiments, a tissue may be studied. For example, a tissue from an organism may be processed to produce cells (e.g., through tissue homogenization or by laser-capturing the cells from the tissue), such that the epigenetic differences within the tissue may be determined, as discussed herein.

The scDNA droplets may further comprise a lysis buffer. In embodiments, the lysis buffer may include, but is not limited to, an enzyme. In some aspects, the enzyme may be any enzyme that is suitable for breaking down DNA into fragments. In examples, the enzyme may comprise micrococcal nuclease (MNase), however, additional enzymes for breaking down DNA may be used as would be understood by those skilled in the art. In some aspects, the lysis buffer may further include a salt, such as, for example, calcium chloride. Without being limited to any particular mechanism or theory, the calcium chloride provides conditions suitable for enzyme digestion of DNA. In some aspects, the concentration of calcium chloride included optimizes enzyme digestion of DNA. In embodiments, the calcium chloride may be present in a concentration of about 0.05 mM to about 0.7 mM, about 0.05 to about 0.6 mM, about 0.05 to about 0.5 mM, about 0.1 mM to about 0.4, or about 0.1 mM to about 0.3 mM.

The method further comprises a step of lysing the single cell or nucleus within each of the plurality of scDNA droplets. In aspects, the lysing step results in a plurality of fragmented scDNA within each of the plurality of scDNA droplets. In some aspects, the fragmented scDNA may have a size ranging from about 50 bp to about 1500 bp, about 60 bp to about 1400 bp, about 70 bp to about 1300 bp, about 80 pb to about 1200 bp, about 90 bp to about 1100 bp, or about 100 bp to about 1000 bp. Beneficially, the scDNA are fragmented and broken down prior to being barcoded and converted via bisulfite conversion, as will be further discussed herein.

In embodiments, the plurality of scDNA droplets are generated at a rate of in the range of about 700 droplets per second to about 4500 droplets per second, about 800 droplets per second to about 4300 droplets per second, about 900 droplets per second to about 4200 droplets per second, or about 1000 droplets per second to about 4000 droplets per second. In other embodiments, the plurality of scDNA droplets are generated at a rate equal to or less than about 4500 droplets per second, less than about 4400 droplets per second, less than about 4300 droplets per second, less than about 4200 droplets per second, less than about 4100 droplets per second, or less than about 4000 droplets per second. In some aspects, the plurality of scDNA droplets each have a diameter in the range of about 10 μm to about 60 μm, about 12 μm to about 55 μm, or about 15 μm to about 50 μm.

The methods and systems of the disclosure further comprise a step of generating a plurality of barcode droplets. In some aspects, each of the plurality of barcode droplets comprise a barcode bead oligonucleotide. In some aspects, the generation step of the plurality of scDNA droplets as described above may occur before the generation step of the plurality of barcode droplets. In other aspects, the generation step of the plurality of scDNA droplets as described above may occur after the generation step of the plurality of barcode droplets. In further aspects, the generation step of the plurality of scDNA droplets as described above may occur before and/or after the generation step of the plurality of barcode droplets.

In some embodiments, barcode bead oligonucleotides are utilized for tagging fragmented scDNA. In some aspects, each of the barcode bead oligonucleotides comprises the following in order from (i) to (iii): (i) a bead for carrying barcode oligonucleotides, (ii) at least one photocleavable linker; and (iii) at least one barcode oligonucleotide sequence. In some aspects, each barcode bead oligonucleotide comprises one bead, with at least one barcode oligonucleotide sequences attached to the same bead, and attached to the bead via the photocleavable linker. In some embodiments, multiple barcode oligonucleotide sequences are attached to the same bead.

In some aspects, the at least one barcode oligonucleotide sequences comprise a PCR priming site, a cell barcode sequence, and a ligation site. In some aspects, the PCR priming site comprises any suitable priming sequence. In some examples, the PCR priming site comprises the sequence CAAGCAGAAGACGGCATACGAGAT (SEQ ID NO: 2). The PCR priming site is further attached to the cell barcode sequence, wherein the cell barcode sequence may have a list of about 10 to about 20 nucleotides (nt) comprising A, G, T, or a combination thereof. For example, the cell barcode sequence may have a list of about 10 nt, 11 nt, 12 nt, 13 nt, 14 nt, 15 nt, 16 nt, 17 nt, 18 nt, 19 nt, or 20 nt. In some exemplary embodiments, the cell barcode sequence may have a sequence of DDDDDDDDDDDDDDD. The cell barcode sequence is further attached to the ligation site, wherein the ligation site may have a list of about 10 to about 50 nucleotides comprising A, G, C, T, or a combination thereof. For example, the ligation site sequence may have a list of 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, or 50 nt. While other ligation site sequences may be used, an example ligation site sequence may include GTCTCGTGGGCTCGGAGATGTGTATAAGAGACAG (SEQ ID NO: 3). In an example, the at least one barcode oligonucleotide sequence may include a sequence of CAAGCAGAAGACGGCATACGAGATDDDDDDDDDDDDDDDGTCTCGTGGGCTCGGAGATGTGTATAA GAGACAG (SEQ ID NO: 1). Example sequences may be found in FIG. 2A.

As further shown in FIG. 2A, a complementary oligonucleotide may be annealed to the ligation site of the at least one barcode oligonucleotide sequence to form a double-stranded tail at a 3′-end of the barcode bead oligonucleotide. In some aspects, the 3′-end of the barcode bead oligonucleotide may be used for ligating to a single fragmented scDNA from the plurality of fragmented scDNA to form the barcoded scDNA within the plurality of barcoded scDNA droplets. In some aspects, the complementary oligonucleotide sequence may include a list of about 15 to about 25 nucleotides. For example, the complementary oligonucleotide sequence may include a list of 15 nt, 16 nt, 17 nt, 18 nt, 19 nt, 20 nt, 21 nt, 22 nt, 23 nt, 24 nt, or 25 nt. For example, the complementary oligonucleotide may comprise a 3′-end blocked complement oligo having a sequence of/5Phos/CTGTCTCTTATACACATCT/3SpC3/(SEQ ID NO: 4), and as shown in FIG. 2A.

In further aspects, the method and systems of the disclosure include a step of fusing the plurality of scDNA droplets and the plurality of barcode droplets to form a plurality of barcoded scDNA droplets. In some aspects, the plurality of barcoded scDNA droplets each comprise a barcoded scDNA as described herein. As shown in FIG. 1, the plurality of barcoded scDNA droplets contain the barcode bead oligonucleotide as well as the plurality of fragmented scDNA. In some aspects, the plurality of barcoded scDNA droplets are exposed to ultraviolet (UV) radiation to remove the bead from the barcode bead oligonucleotides from each of the at least one barcode oligonucleotide sequence via breakage of each of the at least one photocleavable linkers. In some aspects, the UV exposure is provided prior to the incorporation or ligation of the fragmented scDNA to the barcode oligonucleotide sequence to form the barcoded scDNA.

In some aspects, after UV exposure and ligation, the methods and systems of the present disclosure comprise a step of breaking the plurality of barcoded scDNA droplets. In some aspects, the breaking of the plurality of barcoded scDNA droplets allows for preparation of bisulfite conversion of the scDNA. For the bisulfite conversion process, the methods and systems further comprise a step of co-encapsulating the barcoded scDNA with a bisulfite reagent to form a plurality of bisulfite droplets. In some aspects, the bisulfite reagent comprises a bisulfite salt. The bisulfite salt may include, but is not limited to, sodium bisulfite, ammonium bisulfite, and other bisulfite salts suitable for bisulfite conversion. In embodiments, the plurality of bisulfite droplets each have a diameter of about 10 μm to about 250 μm, about 12 μm to about 230 μm, about 15 μm to about 220 μm, about 18 μm to about 210 μm, or about 20 μm to about 200 μm.

The method further comprises a step of converting the barcoded scDNA to bisulfite-converted scDNA within the plurality of bisulfite droplets. Bisulfite treatment of the scDNA can convert unmethylated cytosine residues into uracils (the readout of which can be thymine after amplification with a polymerase). Methylcytosines can be protected from conversion by bisulfite treatment to uracils. Following bisulfite treatment, methylation status of a given cytosine residue can be inferred by comparing the sequence to an unmodified reference sequence. As described previously, in contrast to conventional bisulfite conversion techniques used currently, the methods of the present disclosure provide for the bisulfite conversion of the scDNA within droplets. Beneficially, the conversion of the scDNA within the bisulfite droplets provides for the reduction of DNA degradation. Therefore, the methods of the present disclosure are able to provide for better retention of the scDNA. In some aspects, the plurality of scDNA droplets, the plurality of barcode droplets, the plurality of barcoded scDNA droplets, and the plurality of bisulfite droplets are water-in-oil droplets.

In the example embodiment shown in FIG. 2A, the bisulfite conversion may result in a bisulfite converted PCR priming site, cell barcode sequence, and ligation site having the sequence of UAAGUAGAAGAUGGUATAUGAGATDDDDDDDDDDDDDDDGTUTUGTGGGUTUGGAGATGTGTATAA GAGAUAG (SEQ ID NO: 5), and a bisulfite converted 3′-end blocked complement oligo having the sequence of UTGTUTUTTATAUAUATUT (SEQ ID NO: 6). However, as would be understood by those skilled in the art, other sequences may result depending on the priming site, cell barcode sequence, and ligation site used.

As provided in FIG. 2A, the bisulfite-converted scDNA may further undergo one or more steps comprising random priming and/or indexing PCR for generating the bisulfite sequenced nucleic acid library. For example, the additional steps may comprise random priming with a random primer having a sequence of TCGTCGGCAGCGTCAGATGTGTATAAGAGACAGNNNNNNNNN (SEQ ID NO: 7). The additional steps may further comprise a step of converting the double-stranded DNA (dsDNA) to single stranded DNA (ssDNA) by denaturation resulting in a P5 transposon primer having a sequence of TCGTCGGCAGCGTCAGATGTGTATAAGAGACAG (SEQ ID NO: 8), and a ssDNA pre-PCR sequence having a sequence of CTATCTCTTATACACATCTCCAAACCCACAAAACVVVVVVVVVVVVVVVATCTCATATACCATCTTCTA CTTA (SEQ ID NO: 9). The additional steps may further comprise a step of a pre-amplification process by adding a P5 transposon primer (SEQ ID NO: 8) and P7 transposon BS primer having a sequence of GTCTCGTGGGCTCGGAGATGTGTATAAGAGACAGTAAGTAGAAGATGGTATATGAGAT (SEQ ID NO: 10). This step may further result in a pre-PCR product having a P5 transposon primer and a ssDNA pre-PCR sequence with P7 transposon BS primer having a sequence of CTATCTCTTATACACATCTCCAAACCCACAAAACVVVVVVVVVVVVVVVATCTCATATACCATCTTCTA CTTACTGTCTCTTATACACATCTCCGAGCCCACGAGAC (SEQ ID NO: 11). The sequence may be further complemented prior to indexing PCR. For example, the P5 transposon primer may be complemented by a sequence having a sequence of CTGTCTCTTATACACATCTGACGCTGCCGACGA (SEQ ID NO: 12). The ssDNA pre-PCR sequence with P7 transposon BS primer (SEQ ID NO: 11) may further be complemented with an oligonucleotide having a sequence of

(SEQ ID NO: 13)
GTCTCGTGGGCTCGGAGATGTGTATAAGAGACAGTAAGTAGAAGA
TGGTATATGAGATDDDDDDDDDDDDDDDGTTTTGTGGGTTTGGAG
ATGTGTATAAGAGATAG.

In further embodiments, the one or more steps comprising the indexing PCR may further comprise indexing-PCR-based amplification to generate i5- and/or i7-indexed libraries. For example, as provided in FIG. 2B, the indexing PCR step may further include a N5XX primer having a sequence of AATGATACGGCGACCACCGAGATCTACAC[i5 index] TCGTCGGCAGCGTC (SEQ ID NO: 14 . . . SEQ ID NO: 15). In further embodiments, the indexing PCR step may further include an Ad2.XX primer having a sequence of CAAGCAGAAGACGGCATACGAGAT[i7 index] GTCTCGTGGGCTCGGAGATGT (SEQ ID NO: 2 . . . SEQ ID NO: 16). In a further step, complement oligonucleotides are provided, including a complement of SEQ ID NO: 2 having a sequence of ATCTCGTATGCCGTCTTCTGCTTG (SEQ ID NO: 17) and a complement of SEQ ID NO: 14 having a sequence of GTGTAGATCTCGGTGGTCGCCGTATCATT (SEQ ID NO: 18). After this step, standard sequencing may be completed.

In some aspects, the methods and systems of the disclosure is able to produce a high quantity of single-cell sequencing libraries in a short period of time. The methods and systems of the disclosure provide for the ability to determine the methylation profile of the fragmented scDNA. Beneficially, by utilizing the methods of the disclosed methods and systems, the sequence information from the bisulfite sequenced nucleic acid library can be easily processed as the sequence information with the same barcode oligonucleotide sequence can be identified as being from the same single cell. In embodiments, the methods and systems of the disclosure produce a bisulfite sequenced nucleic acid library of about 500 to about 15,000 single cells, about 600 to about 14,000 single cells, about 700 to about 13,000 single cells, about 800 to about 12,000 single cells, about 900 to about 11,000 single cells, or about 1000 to about 10,000 single cells. In some aspects, the methods and systems of the disclosure may produce the bisulfite sequenced nucleic acid library as described herein within a period of about 5 days, within a period of about 4 days, within a period of about 3 days, or within a period of about 2 days. Beneficially, the provided methods can provide for a bisulfite conversion rate of at least about 90%, at least about 91%, at least about 92%, at least about 93%, at least about 94%, at least about 95%, at least about 96%, at least about 97%, or at least about 98%. In embodiments, the bisulfite conversion rate is at least about 95%.

In some aspects, the methods and systems provided herein take advantage of the rapid speed of droplet microfluidics. Droplet generation and fusion reactions can occur at a speed of hundreds to thousands of droplets per second. Within the methods and systems of the disclosure, millions of droplets can be processed within hours as described herein.

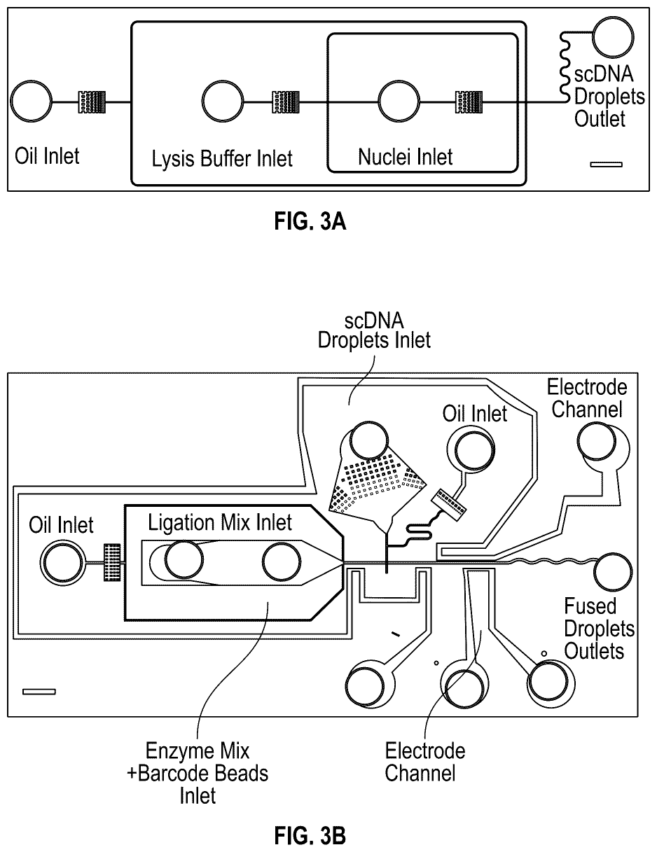

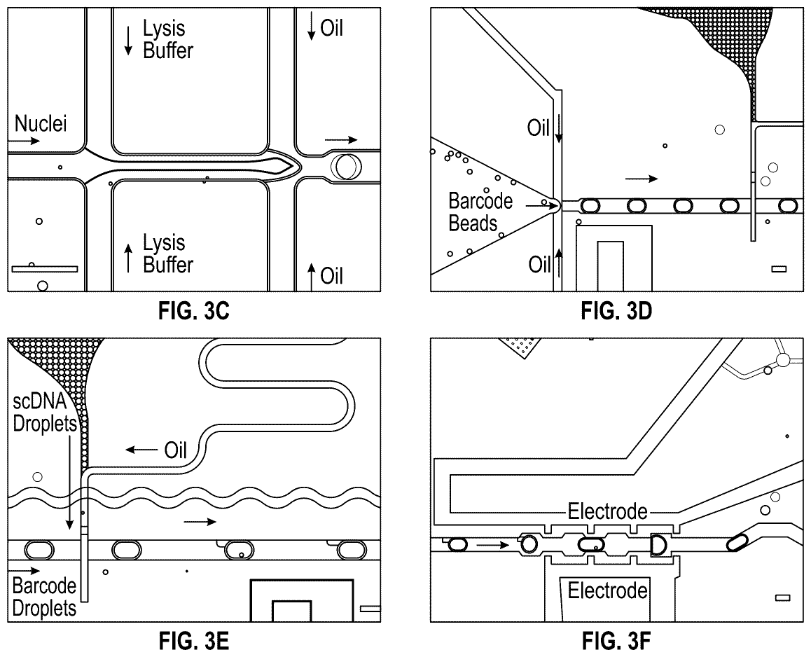

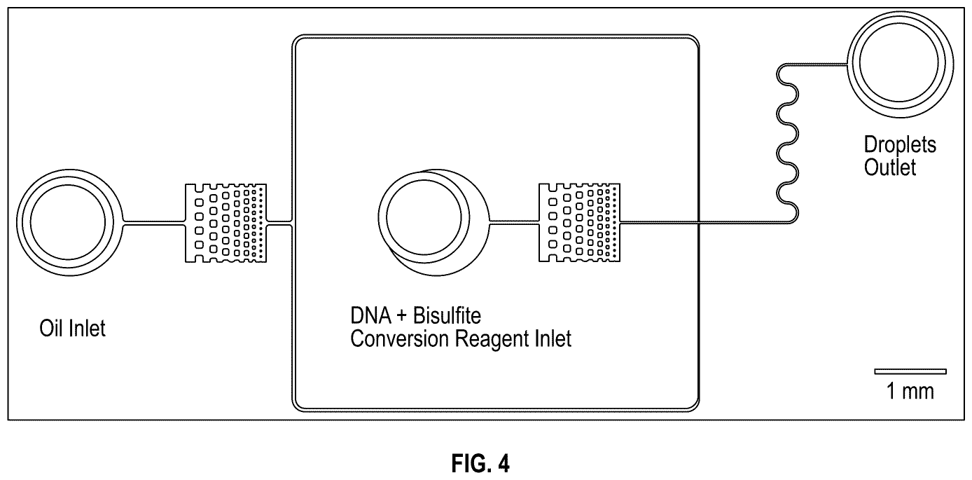

In aspects, the method and systems of the present disclosure may involve devices to generate the bisulfite sequencing library from single cells or nuclei. Examples of the systems suitable for use with the methods provided herein are shown in FIG. 3A, 3B, 3C, 3D, 3E 3F, and FIG. 4. In some aspects, the present disclosure provides for a system for single-cell sequencing. The system comprises a droplet generation device for generating a plurality of scDNA droplets as described herein. For example, the plurality of scDNA droplets may comprise the single cell or nucleus from a population of cells or nuclei as described herein. Further, the plurality of scDNA droplets may comprise a lysis buffer as described herein. An example of the droplet generation device may be found in FIG. 3A. As shown in FIG. 3A, the droplet generation device may be used to encapsulate single nuclei or single cells, as well as the lysis buffer, into droplets having a diameter as already described herein. In embodiments, the droplet generation device may comprise three inlets, including, an oil inlet, a lysis buffer inlet, and a nuclei inlet, and may further include a scDNA droplet outlet to collect produced scDNA droplets. In embodiments, the three separate inlets are each attached to a pump to separately administer the (a) single cell or nucleus from the population of cells or nuclei, (b) the lysis buffer, and (c) an oil, to form the plurality of scDNA droplets. In some example embodiments, the flow rates and concentrations can be adjusted such that only a portion of the droplets contain single cells. In some examples, between about 5% to about 15% of the droplets contain single cells. In further aspects, the probability of having double occupancy of single cells in a droplet can be maintained at a low amount. For example, the probability of having double occupancy of single cells in a droplet is maintained below about 2%, below about 1.5%, below about 1% or below about 0.5%.

In further aspects, the systems of the disclosure provide for a barcode droplet generation unit within a droplet fusion device, wherein the barcode generation unit generates a plurality of barcode droplets. In some examples, the plurality of scDNA droplets may be received in a scDNA droplets inlet within the droplet fusion device prior to forming the plurality of barcoded scDNA droplets. In embodiments, each of the plurality of barcode droplets comprises a barcode bead oligonucleotide as described herein. As shown in FIG. 3B, the plurality of barcode droplets may be produced within the barcode droplet generation unit within a droplet fusion device. The systems further include a droplet fusion device for fusing the plurality of scDNA droplets and the plurality of barcode droplets to form a plurality of barcoded scDNA droplets, each comprising a barcoded scDNA. As further shown in FIG. 3B, the barcode droplets are produced and then paired or fused with reinjected scDNA droplets. In some aspects, an average of about 60% to about 95%, about 70% to about 90%, or about 75% to about 85% of the barcode droplets may be fused with the scDNA droplets.

In some aspects, the droplet fusion device further comprises one or more electrode channels comprising a salt solution for conducting dielectrophoresis-based droplet fusion to fuse the plurality of scDNA droplets and the plurality of barcode droplets to form the plurality of barcoded scDNA droplets. In examples, there are two channels that are filled with salt solution and serve as electrodes for conducting dielectrophoresis-based droplet fusion. In embodiments, one of the electrode channels surrounds most of the microfluidic structure, acting as a shield field to prevent reinjected droplets from coalescence. In further embodiments, filtration structures may be placed between the inlets and the narrow channels to trap particles and prevent clogging. In some aspects, the depth, as well as the width, of the side channel for re-injection of scDNA droplets may be in the range of about 15 μm to about 45 μm, about 20 μm to about 40 μm, about 25 μm to about 35 μm, or around 30 μm, for avoiding multi-layer stacking of the droplets and avoiding simultaneous release of multiple scDNA droplets during fusion. In other embodiments, the depth of the rest of the device may be in the range of about 55 μm to about 85 μm, about 60 μm to about 80 μm, about 65 μm to about 75 μm, or about 70 μm, to avoid clogging by barcode bead droplets containing beads of about 20 μm to about 40 μm in diameter.

An exemplary example of the process is further described and shown in FIG. 3C to FIG. 3F. The figures show microscopic images of the devices of the disclosure. In FIG. 2C, the figure shows the devices in operation for single-nuclei or single cell droplet generation. In FIG. 2D, the barcode droplets are generated in the droplet fusion device. FIG. 2E shows the reinjection of scDNA and pairing with the barcode droplets. Further, FIG. 2F shows the fusion of scDNA droplets and the barcode droplets under dielectrophoresis.

In further embodiments, a bisulfite droplet device is used for co-encapsulating the barcoded scDNA with a bisulfite reagent to form a plurality of bisulfite droplets as described herein. In an example embodiment, a bisulfite droplet device may be as shown in FIG. 4 to generate the plurality of bisulfite droplets as described herein. As shown in FIG. 4, the bisulfite droplet device may include an oil inlet, a scDNA and bisulfite conversion reagent inlet, and a droplet outlet. In some embodiments, filtration structures may be placed between the inlets and the narrow channels to trap particles and prevent clogging.

In comparison to competing technologies, the methods of the present disclosure (Drop-BS) offer increased throughput owing to the advantage of the droplet-based processes platform. Therefore, the methods of the disclosure is able to provide much higher throughput in comparison to conventional single-cell bisulfite sequencing techniques, such as snmC-seq, sciMET, sciMETv2, and scBS-seq, while maintaining bisulfite conversion rates greater than 95%.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Materials and Methods

A number of materials and methods were used in evaluating the droplet-based single-cell bisulfate sequencing (Drop-BS) methods and systems as described within the disclosure. This example provides further details regarding exemplary example embodiments of the Drop-BS method and system of the disclosure.

Cell Culture: Throughout the Examples, various cell cultures were evaluated. GM12878 cells (lymphoblastoid cell line, LCLs) were purchased from Coriell Institute and propagated in RPMI 1640 (ATCC) containing 15% fetal bovine serum, 100 U/mL Penicillin-Streptomycin (containing 100 units/mL of penicillin and 100 μg/mL of streptomycin) at 37 °C in a humidified incubator containing 5% CO₂. GM12878 cells were sub-cultured every two days. Further, HEK293 cells (human embryonic kidney cells) and MCF7 cells (human breast cancer cells) were purchased from ATCC and cultured in DMEM (high glucose) containing 15% fetal bovine serum, and 100 U/mL Penicillin-Streptomycin at 37 °C in a humidified incubator containing 5% CO₂. The HEK293 cells and MCF7 cells were sub-cultured when ~80% confluency was reached.

Mouse Brain Samples: C57BL/6J mice were purchased from Jackson Laboratory and maintained with 12-hour light/12-hour dark cycles, with food and water provided ad libitum. Ten-week-old male mice were sacrificed by compressed CO₂ followed by cervical dislocation. Mouse brains were rapidly dissected, frozen on dry ice, and stored at −80 °C. To collect prefrontal cortex (PFC) samples, mouse brains were sectioned coronally at the bregma (1.90 to 1.40 mm) with a razor blade in dissection media (20 mM sucrose, 28 mM D-glucose, and 0.42 mM NaHCO₃ in HBSS). The dissected brain tissues were stored at −80 °C until use. The Examples utilizing the mouse brain samples were approved by the Institutional Animal Care and Use Committee (IACUC) at Virginia Tech.

Human Brain Samples: Human brains were obtained during autopsies performed at the Basque Institute of Legal Medicine, Bilbao, Spain. The Examples utilizing human brain samples were developed in compliance with policies of research and ethical review boards for post-mortem brain studies (Basque Institute of Legal Medicine, Spain). Specimens of the frontal cortex (Brodmann area 9) were dissected at autopsy (0.5-1 g tissue) on an ice-cooled surface and immediately stored at −80 °C until use. Tissue pH values were within a relatively narrow range (6.7±0.08). Brain samples were also assayed for RNA integrity number (RIN) values (7.87±0.21). The brain tissue for the human brain 1 (HB1) sample used in the Examples belonged to a 41-year-old Caucasian female, and the brain tissue for the human brain 2 (HB2) sample used in the Examples belonged to a 74-year-old Caucasian female with a postmortem interval (PMI) of 22 hours, with negative medical information on the presence of neuropsychiatric disorders or substance use disorder, and negative toxicological screening results for either psychotropic drugs or ethanol.

Microfluidic Device Development: Three microfluidic devices were designed and fabricated for use in the methods and systems disclosed using soft lithography, including a droplet generation device, a droplet fusion device, and a bisulfite droplet device. The channel structures for each device were designed using LayoutEditor and printed on a transparency photomask with high resolution (10,000 dots per inch, Fineline imaging). Molding of the droplet generation device (FIG. 3A) and the bisulfite droplet device (FIG. 4) were developed using one photomask for each. Further, molding for the droplet fusion device (FIG. 3B) was developed using two photomasks. The devices were molded in polydimethylsiloxane (PDMS). Degassed PDMS prepolymers mixed with catalyst at a ratio of 10:1 (w/w) was poured onto a master, degassed again, and cured at 80 °C for 1.5 hours. The cured PDMS slab with an about 5 mm thickness was slowly peeled from the master. Inlet and outlet holes were punched into the PDMS slab. The PDMS slab and a pre-cleaned glass slide were then placed in a plasma cleaner and treated for 1 minute. The oxidized PDMS and glass surfaces were brought into contact and bonded to form a permanent seal after baking at 80 °C for 24 hours. The microfluidic devices were rendered hydrophobic prior to use by treating with Aquapel (Aquapel Glass Treatment, PGW Auto Glass, LLC) for 2 minutes followed by baking at 80 °C for 10 min.

Nuclei Extraction from Mouse/Human Brain Tissues: To isolate nuclei, the PFC was placed in 5 ml ice-cold nuclei extraction buffer (0.32 M sucrose, 5 mM CaCl₂), 3 mM Mg(Ac)₂, 0.1 mM EDTA, 10 mM Tris-HCl, and 0.1% Triton X-100, with freshly added 50 μl of PIC (protease inhibitor cocktail), 5 μl of 100 mM PMSF (phenylmethanesulfonyl fluoride) and 5 μl of 1 M DTT (dithiothreitol)). The tissue was thawed and homogenized by slowly douncing on ice with a loose pestle A (Kimble, 7-ml size) for 15 times, and with a pestle B for 25 times. The homogenate was filtered with a 40-μm nylon mesh cell strainer and then the sample was centrifuged at 1000 g for 10 min at 4 °C. The supernatant was then removed and the pellet resuspended in 500 μl of ice-cold nuclei extraction buffer.

The suspension was mixed with 750 μl of 50% iodixanol solution (50% iodixanol, 25 mM KCl, 5 mM MgCl₂, and 20 mM Tris-HCl (pH 7.8)) and centrifuged at 5000 g for 15 min at 4 °C. After centrifugation, a s3 to single-cell whole genome sequencing (s3_WGS) protocol was followed to perform nuclei fixation and nucleosome depletion. The nuclei pellet was resuspended in 5 ml of NIB:HEPES buffer (10 mM HEPES-KOH (pH 7.2), 10 mM NaCl, 3 mM MgCl₂, 0.1% Igepal, 0.1% Tween-20) in a 15 ml centrifuge tube. The mixture was incubated at room temperature for 10 min on a rotator (speed: 50 rpm) after adding 246 μl of 16% formaldehyde to the suspension. The mixture was centrifuged at 500 g for 5 min at 4 °C to form nuclei pellet and the supernatant was then removed. 1 mL of NIB:Tris (10 mM Tris-HCl (pH 7.4), 10 mM NaCl, 3 mM MgCl₂, 0.1% Igepal, 0.1% Tween-20) was then added to resuspend and wash the nuclei pellet to quench the fixation. The nuclei were pelleted by centrifuging at 500 g for 5 min at 4 °C and resuspended in 200 μl 1×NEBuffer 2.1 (50 mM NaCl, 10 mM Tris-HCl, 10 mM MgCl₂, and 100 μg/ml BSA) in a 1.5 ml tube. The suspension was centrifuged at 500 g for 5 min at 4 °C to pellet nuclei which were then resuspended in 760 μl of 1×NEBuffer 2.1. Next, 40 μl of 1% SDS was added to the nuclei suspension and the mixture incubated at 37 °C for 20 min on a Multi-Therm shaker (Benchmark Scientific, H5000-HC) with a shaking speed of 300 rpm. After SDS treatment, the mixture was centrifuged at 500 g for 5 min at 4 °C. The pellet was resuspended in 1 ml of ATAC-RSB buffer (10 mM Tris-HCl (pH 7.4), 10 mM NaCl, 3 mM MgCl₂ with freshly added 10 μl of 10% Tween-20). The suspension was pipetted up and down several times and then centrifuged at 500 g for 5 min at 4 °C. Lastly, the nuclei were resuspended in 100 μl of ATAC-RSB buffer with freshly added 1 μl of 10% Tween-20. The concentration of the nuclei was measured using a hematocytometer and the nuclei concentration diluted to 5 million/ml using the ATAC-RSB buffer.

Nuclei Extraction from Cell Lines GM12878, HEK293, and MCF7 cells: 1 ml of suspended cells were centrifuged at 300 g for 5 min at 4 °C. Cells were then resuspended in 500 μl ice-cold nuclei extraction buffer. The rest of the procedure was identical to the process described above for brain tissues.

Design and Preparation of Barcode Beads: Barcode beads with customized oligonucleotide sequences were purchased from ChemGenes (Wilmington, MA). Specifically, each oligonucleotide on the bead surface consisted of four parts (FIG. 2A): (1) a photo-cleavable linker that connects the oligonucleotide to the bead surface and releases the oligonucleotide after UV exposure; (2) a PCR priming site (identical for all beads; CAAGCAGAAGACGGCATACGAGAT (SEQ ID NO: 2)); (3) a 15-nucleotide barcode sequence comprising A, T, or G bases for labeling single cells (unique for each bead; DDDDDDDDDDDDDDD); and (4) a ligation site (GTCTCGTGGGCTCGGAGATGTGTATAAGAGACAG (SEQ ID NO: 3)).

To form a double-stranded tail for ligation, a complimentary 19 nt-oligonucleotide (referred to as “3′-end blocked complement oligo” with the sequence as shown in FIG. 2A) was annealed to the bead oligonucleotides. About 20,000 barcode beads were washed three times with 0.5 ml of TE-TW (10 mM Tris pH 8.0, 1 mM EDTA, and 0.01% Tween-20). 70 μl oligo annealing buffer (10 mM Tris pH 8.0, 50 mM NaCl, and 1 mM EDTA) and 30 μl of 100 μM complimentary oligonucleotide were mixed, and the barcode beads were resuspended in the mixture. The mixture was incubated at 85 °C with a shaking spend of 1500 rpm for 5 min on a Multi-Therm shaker (Benchmark Scientific, H5000-HC). The mixture was slowly cooled down to room temperature while continuing to shake the mixture at 1500 rpm. The prepared barcode beads were washed with TE-TW three times prior to loading into the microfluidic device.

Single-Nuclei Lysis and DNA Fragmentation in Droplets: In a typical experiment, the below protocol was used to generate bisulfite sequencing libraries on about 2,000 single cells. Three 0.5 m-long PFA tubing connected to three syringes were loaded with 100 μl of the nuclei suspension, 160 μl of 2× lysis buffer (2% Triton x-100, 100 mM Tris-HCl pH 8.0, 300 mM NaCl, 0.2% sodium deoxycholate, 10 mM sodium butyrate, 0.375 mM CaCl₂, 0.0375 U/μl MNase, 0.2% Sarkosyl, with freshly added 1.6 μl of PIC and 1.6 μl of 100 mM PMSF), and 1 ml oil (Bio-Rad, 1864006), respectively. The three syringes were mounted on three separate syringe pumps (Chemyx, FUSION 200) and the reagents were flowed simultaneously into the three inlets of the droplet generation device (FIG. 3A) at flow rates of 2 μl/min for the nuclei suspension, 2 μl/min for the lysis buffer, and 14 μl/min for the oil to generate droplets with a size of about 35 μm in diameter. The generated droplets were continuously collected into a 0.2 ml tube that was placed on ice for 10 min to gather about 1.78 million droplets. The droplets were incubated on ice for 10 minutes, then at room temperature for 15 minutes, and finally at 65 °C for 30 minutes. After incubation, excessive oil at the bottom of the tube was removed and the tube was put on ice until use.

Single-Cell DNA Barcoding in Droplets: The electrode channels (FIG. 3B) in the droplet fusion device were filled with 2 M NaCl solution and connected to a high voltage alternating current (AC) power supply with an output set at 300 Vp-p and 200 kHz (Micromechatronics, PDUS210-800V). Single-cell droplets generated after cell lysis and DNA fragmentation were re-injected into the droplet fusion chip (FIG. 3B) at a flow rate of 0.12 μl/min. About 20,000 prepared barcode beads were resuspended in 40 μl enzyme buffer (1 U/μl Fast-Link DNA Ligase (Lucigen, E0077-2-D3), 20% End-It Enzyme Mix (Lucigen, E0025-D1), and 18% Glycerol) and flowed into the chip at a flow rate of 1.1 μl/min. 150 μl of ligation mix (1.5× Fast-link ligation buffer (Lucigen, SS000272-D5), 26.7 mM EGTA (bioWorld, 40520008-2), 1.3 mM dNTPs (Thermo Fisher Scientific, R0194), 1.2 mM ATP (Lucigen, SS000391-D3), and 16% Glycerol (Sigma-Aldrich, 56-81-5)), 1 ml spacing oil, and 1 ml droplet generation oil were flowed into the device with flow rates of 3 μl/min, 3 μl/min, and 7 μl/min, respectively. Merged droplets were collected into a 1.5 ml tube on ice. The droplets were then exposed to 365 nm UV exposure (160 mJ/cm²) for 8 minutes and incubated at 16 °C for overnight (12-18 hours). The droplets were further incubated at 70 °C for 15 minutes for the inactivation of ligase and end-it enzyme and then broken by adding 100 μl of 100% perfluorooctanol. 15 μl of 20 mM concentration of Proteinase K was added to the extracted aqueous phase (about 150 μl) and incubated at 65 °C for 2 hours. DNA was first purified using 1.4× SPRIselect bead (Beckman Coulter Life Sciences, B23317) and selected using 1× SPRIselect bead. Finally, DNA was eluted into 20 μl low-EDTA TE buffer (10 mM Tris, 0.1 mM EDTA).

Bisulfite Conversion in Droplets: Barcoded DNA in 20 μl low-EDTA TE buffer was heated at 95 °C for 1.5 min and immediately put on ice for 2 min to produce single-stranded DNA. The DNA solution was then mixed with 130 μl bisulfite conversion reagent (Promega, N1301, MethylEdge® Bisulfite Conversion System) before being loaded into the bisulfite droplet device (FIG. 4) at a flow rate of 5 μl/min, with the oil's flow rate at 15 μl/min. The collected bisulfite droplets were incubated at 95 °C for 5 min and 54 °C for 1 hour for bisulfite conversion in droplets. Droplets were then broken to separate the aqueous phase into a new tube. DNA desulphonation and cleanup steps were immediately performed following the manufacturer instructions of the MethylEdge® Bisulfite Conversion System kit from Promega. After the column cleanup, DNA was selected by 1.1× SPRIselect bead and eluted into 11 μl low-EDTA TE buffer. The bisulfite conversion rate was examined using unmethylated lambda phage DNA (Promega).

Library Preparation: A number of steps were involved in the sequencing library preparation. Non-limiting exemplary steps utilized in the Examples of the disclosure included the following.

Random Priming—Treated DNA (11 μl) was mixed with 1 μl of 50 μM random primer (with the sequence shown in FIG. 2A) and heated at 95 °C for 3 min and immediately put on ice for 2 min to produce single-stranded DNA. The solution was subsequently mixed with 2 μl blue buffer (10×, Enzymatics, P7010-HC-L), 1 μl of 50 U/μl Klenow exo-(Enzymatics, P7010-HC-L), and 5 μl of 10 mM dNTPs on ice and incubated at 4 °C for 5 minutes, then a slow increase of 0.1 °C/second to 25 °C, 25 °C for 5 min, then a slow increase of 0.1 °C/second to 37 °C, and 37 °C for 60 min. DNA was then purified using 1.1× SPRIselect bead and then incubated at 95 °C for 1.5 min and immediately put on ice for 2 minutes to generate single-stranded DNA, followed by another round of purification using 1.1× SPRIselect bead. 19.5 μl of low-EDTA TE was used to elute DNA from the SPRIselect beads.

Pre-Amplification—The 19.5 μl eluate was placed in a 0.2 ml PCR tube preloaded with a PCR reaction mixture containing 25 μl of 2× KAPA HiFi HotStart Uracil+ReadyMix (Roche, KK2801), 1.5 μl of 25 μM P5 transposon primer (with the sequence shown in FIG. 2B), 1.5 μl of 25 μM P7 transposon BS primer (with the sequence shown in FIG. 2B), and 2.5 μl DMSO (Sigma, D9170-5VL). The PCR tube was put on a thermocycler and incubated with the program of 98 °C for 30 seconds, (98 °C for 10 seconds, 58 °C for 30 seconds, 72 °C for 30 seconds) for 5 cycles, and a final 72 °C extension for 1 minute. After PCR, DNA was purified using 1× SPRIselect beads and eluted into 19.5 μl low-EDTA TE.

Indexing PCR Amplification—The 19.5 μl eluate was placed in one microwell of a 96-well plate preloaded with a PCR reaction mixture containing 25 μl of 2× KAPA HiFi HotStart Uracil+ReadyMix (Roche, KK2801), 1.5 μl of 25 μM N5XX primer (with the sequence shown in FIG. 2B), 1.5 μl of 25 μM Ad2.XX primer (with the sequence shown in FIG. 2B), and 2.5 μl EvaGreen dye (20×, Biotium, 31000). The 96-well plate was put in a Bio-Rad CFX thermocycler and incubated with the program of 98 °C for 30 seconds, a program of 98 °C for 10 seconds, 62 °C for 30 seconds, and 72 °C for 30 seconds, for 3 cycles to achieve >200 RFU increase, followed by a final 72 °C extension for 1 minute. After PCR, DNA was purified using 0.85× SPRIselect bead and eluted into 20 μl low-EDTA TE.

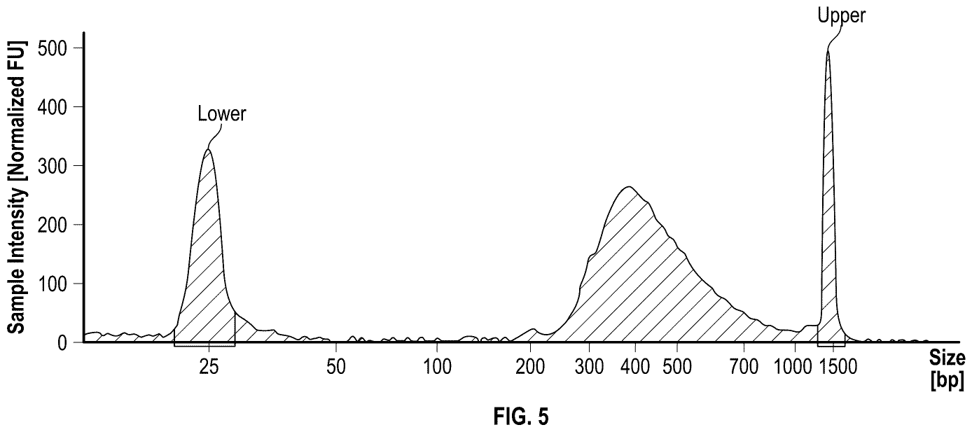

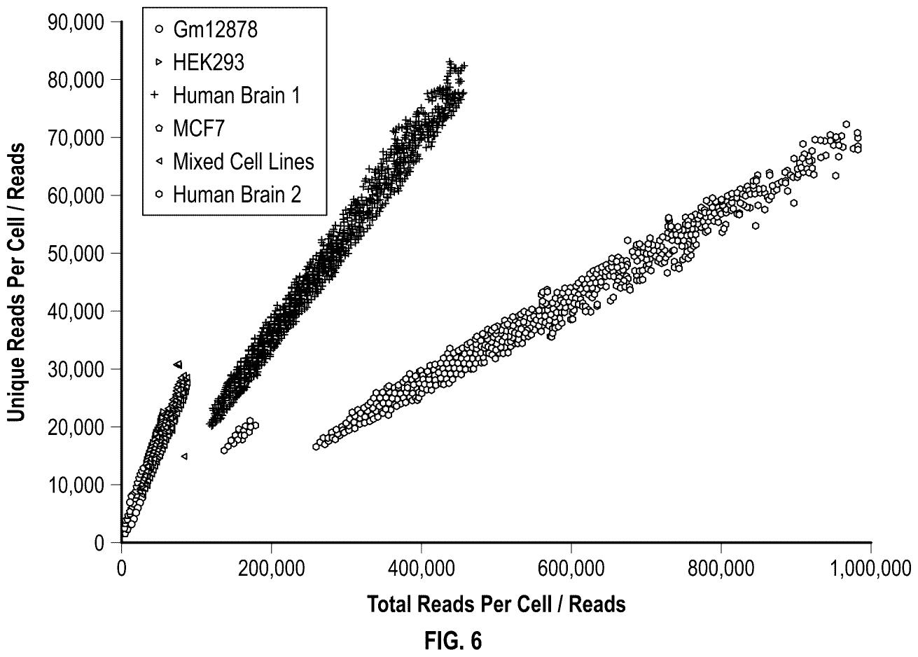

Library Quantification and Sequencing: As shown in FIG. 5, the size of the library of 2000 cells was measured using a High Sensitivity D1000 ScreenTape (Agilent Technologies) on a TapeStation (Agilent Technologies). The concentration of the library was quantified using a KAPA Library Quantification Kit (Roche). The library mixed with 5% PhiX or other high-complexity libraries was loaded on a S4 lane of NovaSeq 6000 system. The sequencing followed the standard Illumina protocol with 150 bp paired-end reads. The average number of unique reads per cell generally increased with higher assigned number of reads first until it plateaued (FIG. 6).

Sequencing Data Analysis: A number of analyses were performed on the sequencing data generated. Non-limiting exemplary analyses utilized in the Examples of the disclosure included the following.

Raw Read Demultiplexing Based on Barcodes—Single-cell Drop-BS data were extracted from raw sequencing reads based on the barcode sequence. A barcode whitelist including the barcodes associated with the most raw reads was identified using umi_tools (v1.0.1) whitelist. Sequencing reads with a specific barcode from the barcode whitelist were extracted using umi_tools extract. Sequencing reads were trimmed to remove artificial bases introduced by end repair during ligation reaction. Trimmed reads were sorted into individual files based on the barcodes using idemp. Alignment to the hg19/mm10 reference genome was performed using Bismark (v0.23.0, bowtie2 (v2.3.5.1)) with the default settings for R2 reads and—pbat for R1 reads (R1 and R2 reads were processed separately).

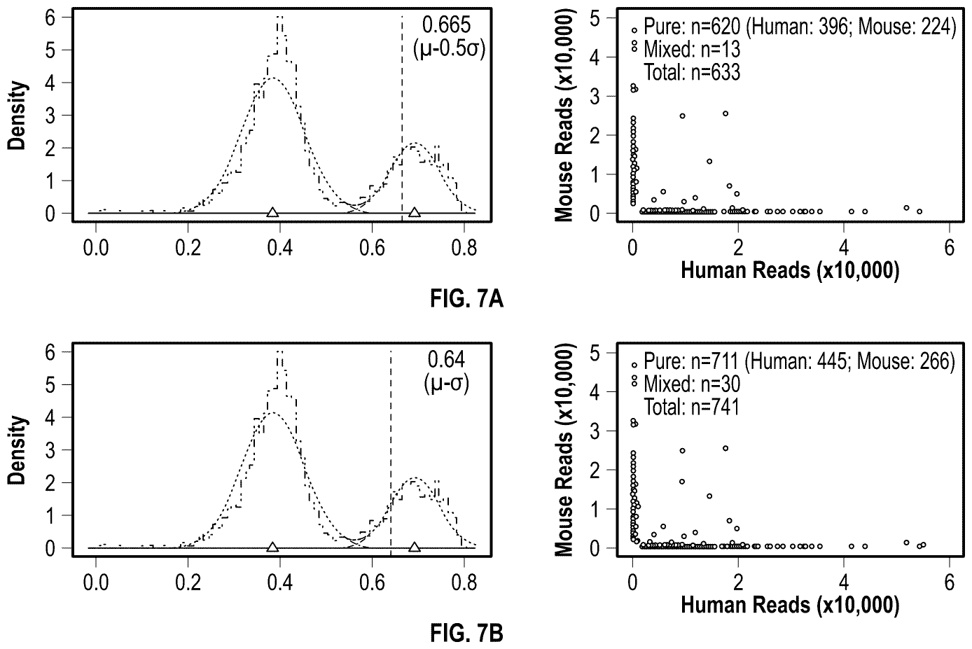

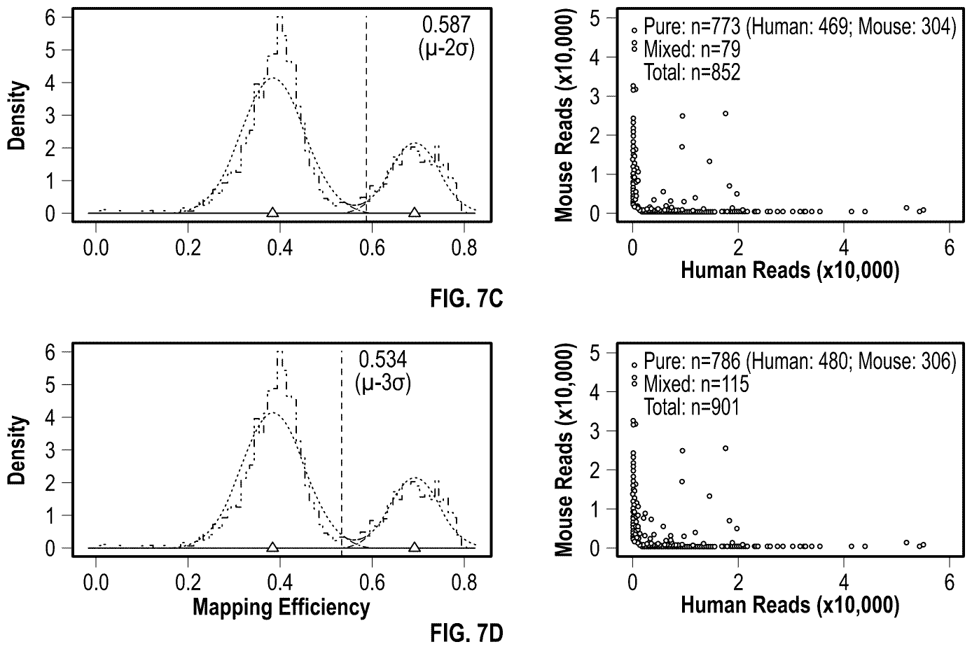

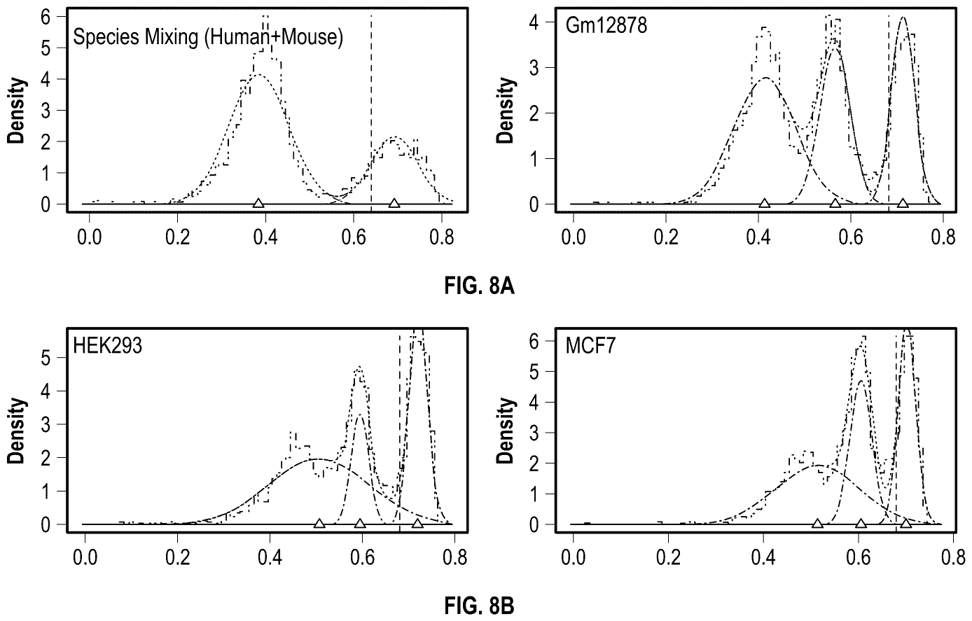

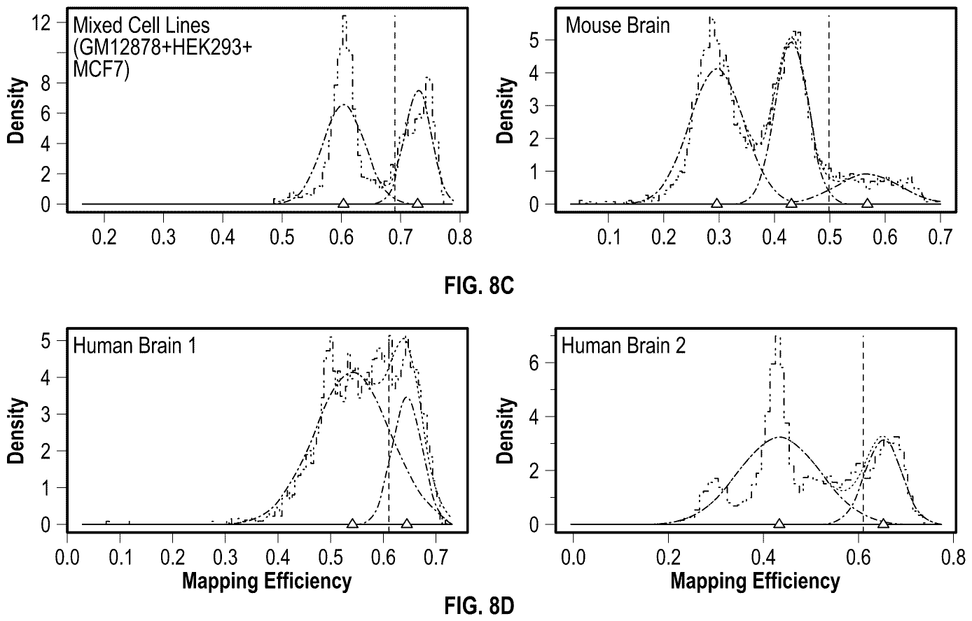

Selection of Cell-Associated Barcodes—2.5× barcodes (X=the number of cells expected based on experimental parameters) were selected with the most raw reads. A probability density plot was created to show the distribution of mapping efficiency for all selected barcodes (see FIG. 7A to FIG. 7D and FIG. 8A to FIG. 8D). Multiple normal distributions were used to fit the density plot. The normal distribution with the highest mean mapping efficiency was considered as having the cell-associated barcodes. In order to remove the potential overlap with the adjacent distribution, the value of “μ-σ” was used as the cutoff for mapping efficiency. The mapping efficiency cutoff was specific to each specific dataset.

Quality Control and Construction of allc File for Each Single Cell/Barcode—R1 and R2 reads for each single barcode were combined into one file using bamtools merge. Picard was used to remove duplicate reads for each barcode. The methylation state for each cytosine was then identified in the unique reads.

Construction of Feature Matrix and Clustering—allc files were used to calculate the methylation rate (mCG/CG or mCH/CH) over bins (1 Mb for cell line data to match the low sequencing depth, or 100 kb for the mouse/human brain data to facilitate comparison with previous work) across the genome for each single cell. The methylation rate data was combined from all singe cells into a feature matrix. Single-cell data in the matrix were then normalized by dividing by the global mCG/CG or mCH/CH of the specific single cell. Principal component analysis (PCA using the irlba package (v 2.3.5) in R) was conducted to reduce the dimensions of the normalized matrix to the top 50 principal components for Louvain clustering (using the igraph package (v1.3.1)). The clustering results were projected using uniform manifold approximation and projection (UMAP (v 0.2.8.0)).

CG Methylation Z-Score Calculation—Unique reads of single cells were merged within each cluster. The CG methylation level of each cluster was calculated over previously defined neuron type specific DMRs. Scale function in R was used to calculate the methylation z-scores and plotted the data using pheatmap (v1.0.12) in R.

Pseudobulk DMR Analysis—Single-cell data within the excitatory neuronal clusters (cluster 1, 4, and 5) from Human Brain 1+2 were merged to generate the excitatory neuronal pseudobulk data. Similarly, the inhibitory neuronal pseudobulk data were produced from the inhibitory neuronal cluster (cluster 3). In these cases, all “allc_*.tsv” files were combined from the excitatory or inhibitory neuronal cells into one “allc_*.tsv” file. For each unique CpG in the combined file, the total number of methylated cytosines and the total number of cytosines were calculated. This information was used as the pseudobulk data for downstream DMR calling. DSS package (v2.48.0) in R was used to call CG-DMRs between the excitatory neuronal data and the inhibitory neuronal data. ChlPseeker package (v1.36.0) was further used in R to annotate the DMRs to genes.

Data Availability: The Drop-BS data on cell lines and mouse brain and the processed Drop-BS data on human brain can be accessed via Gene Expression Omnibus (GEO) under accession number GSE 204691. The raw Drop-BS data on human brain were deposited in dbGaP under accession number phs002123.v2.p1.

### Example 2

### Characterization of Drop-BS Performance

Within this example, a species-mixing experiment was performed to assess the purity of the single-cell methylomic data generated by Drop-BS as described in Example 1. GM12878 nuclei (a human cell line) and mouse brain nuclei were mixed at a 1:1 ratio and a single-cell bisulfite sequencing library of ~1,000 cells were prepared according to the methods described in Example 1. After selection of cell-associated barcodes (see FIG. 7A to 7D), 741 high quality barcoded single-cell data were recovered, where a vast majority (~96%) of which had >90% of their reads aligned to human hg19 genome (n=445) or mouse mm10 genome (n=266). The data demonstrated that the Drop-BS platform of the disclosure effectively limited crosstalk across droplets and produced high-purity single-cell datasets.

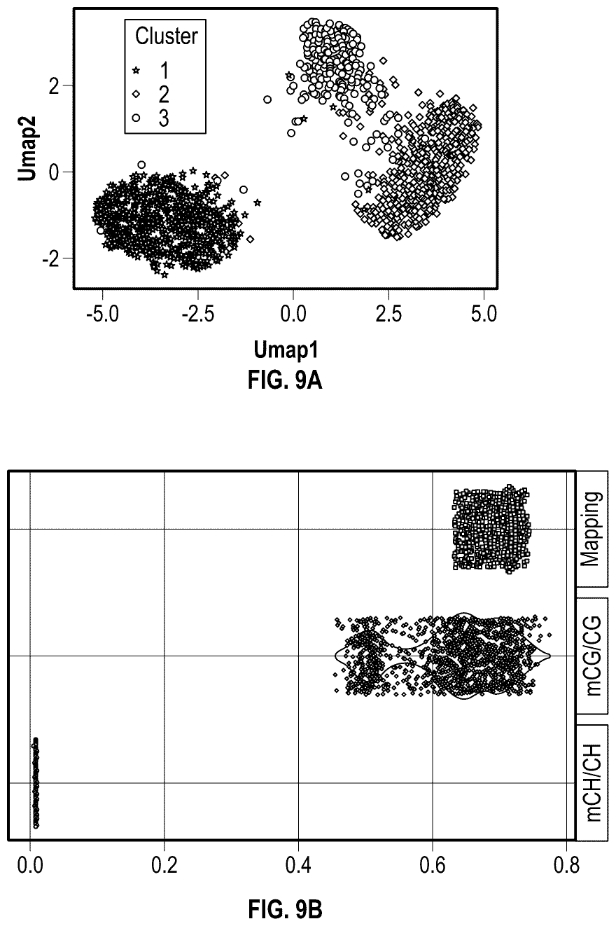

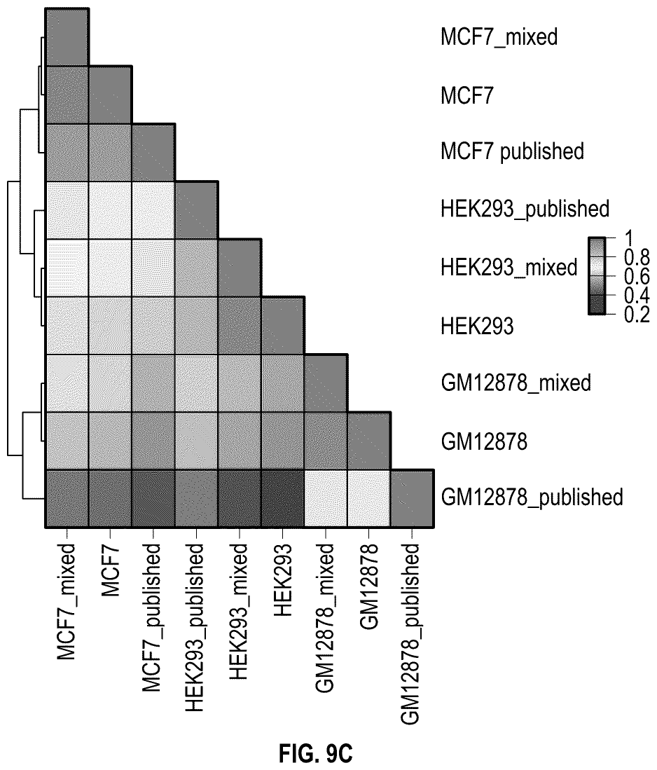

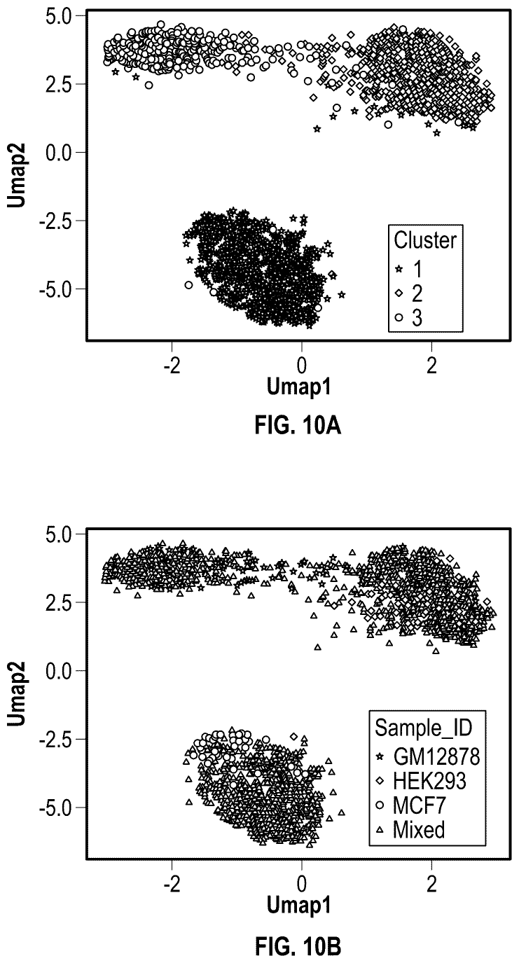

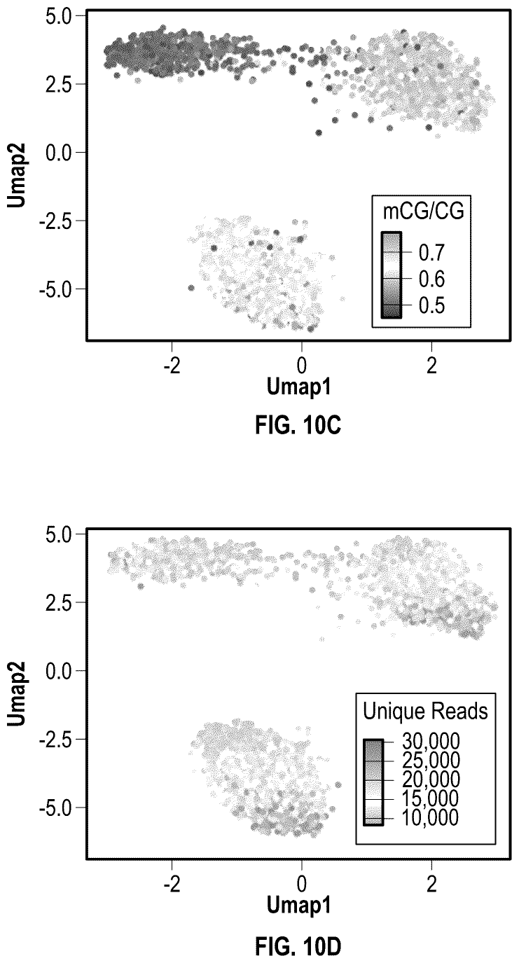

Drop-BS libraries were prepared using three cell lines of different types as described within Example 1, as well as a mixture of the three cell lines in equal portions. High-quality single-cell data were selected for each sample. The samples included GM12878 (1060 cells), HEK293 (1489 cells), MCF7 (1263 cells), and mixed (1929 cells) (see FIG. 8A to 8D). UMAP and Louvain clustering were performed on the methylomic data of the mixture (averagely ~16,157 unique reads per cell for 1929 cells) and obtained three clusters (FIG. 9A). Co-clustering was further conducted by adding 300 single-cell data (100 for each cell line) with known identities (FIGS. 10A and 10B) to confirm the assignment of the clusters to specific cell lines. It was shown that the UMAP clustering was due to different mCG levels associated with the three cell types as shown in FIG. 10C, instead of the number of unique reads per cell as shown in FIG. 10D. However, the unevenness in the number of unique reads among single cells created spread within the clusters and potentially decreased the resolution of various cell types when they were similar (FIG. 10D). The average global mCG level for each cell type reflected by the Drop-BS data (50.5% for the GM12878 cluster, 68.2% for the HEK293 cluster, and 63.0% for the MCF7 cluster) (FIG. 9B) were similar to previously reported values by bulk assays (48% for GM12878, 66% for HEK293, and 65% for MCF7). Merged single-cell data of the clusters had high correlation with their corresponding merged single-cell data of individually profiled cell lines and previously published methylomic data on the cell lines (FIG. 9C).

### Example 3

### Drop-BS Profiling of Brain Samples

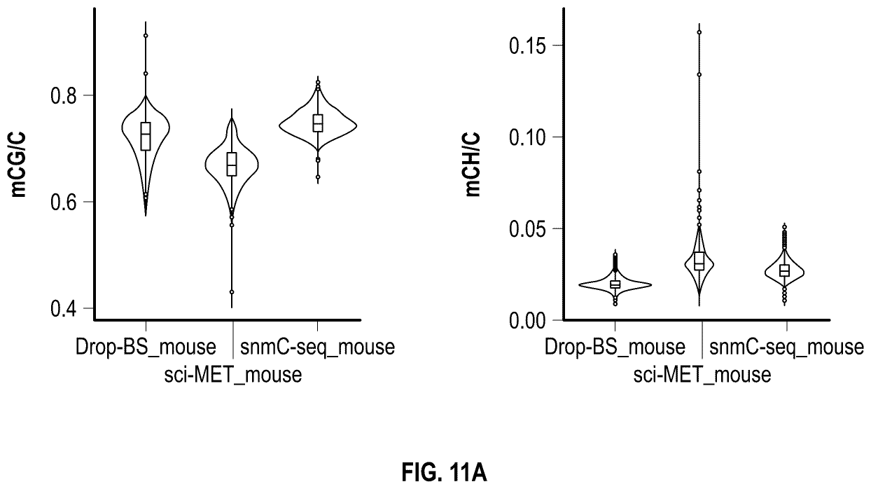

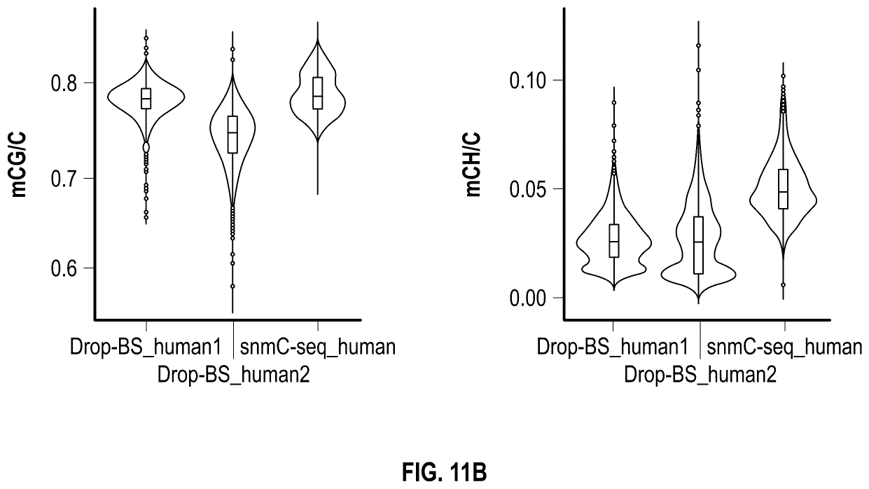

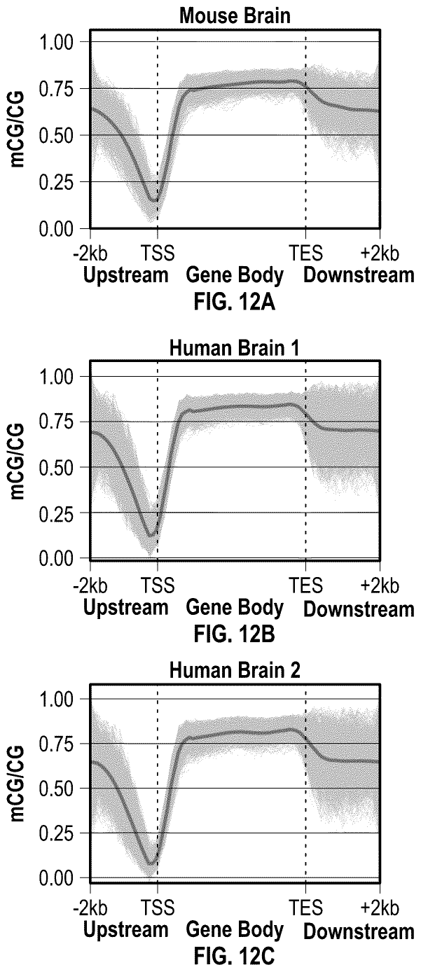

Within this Example, the Drop-BS technology of the disclosure was applied to study prefrontal cortex (PFC) samples from mice and humans. The brain represents the most complex organ and contains a complex mixture of various cell types. The range of CG and CH methylation levels in the Drop-BS data on the mouse and human PFCs were comparable to previously published data, as shown in FIGS. 11A and 11B, and Table 1. The Drop-BS data revealed an average global mCG/CG of 71.73% and 76.12%, mCH/CH of 1.85% and 2.71%, for mouse and human PFCs respectively. The high mCH levels in the brain samples, compared to 1.0% in GM12878 cells, 1.1% in HEK293 cells, and 0.9% in MCF7 cells, were in agreement with previous literature. Evident hypomethylation regions around TSS and higher methylation level throughout the gene body than that of adjacent intergenic regions were further observed as shown in FIG. 12A, FIG. 12B, and FIG. 12C.

TABLE 1

| | DropBS | sci-MET | snmC-seq | DropBS | DropBS | snmC-seq |
|---|---|---|---|---|---|---|
| | mouse | mouse brain | mouse brain | human | human | human brain |
| | brain data | data | data | brain 1 data | brain 2 data | data |
| mCG/CG | 71.73 ± 3.77% | 65.97 ± 3.82% | 74.49 ± 2.34% | 77.99 ± 2.07% | 73.80 ± 3.39% | 78.71 ± 2.28% |
| mCH/CH | 1.85 ± 0.39% | 2.73 ± 1.50% | 2.50 ± 0.49% | 2.70 ± 1.16% | 2.72 ± 1.68% | 5.09 ± 1.44% |

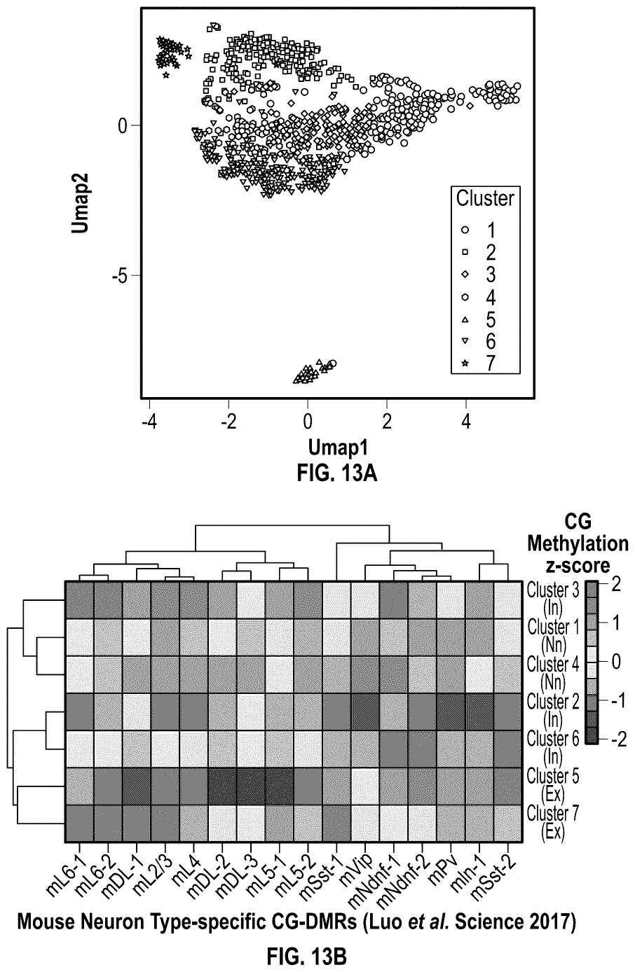

After discarding low-quality barcodes (See Example 1), 1123 single-cell mouse PFC methylome were generated with averagely 23,932 unique reads per cell and an average mapping efficiency of ~58%. On average, 13,492 CpGs were covered per cell and a total of 11,421,613 CpGs were covered by the merged 1123 single-cell data. Louvain clustering of single cell methylomes were further conducted based on their CH methylation level over genome-wide 100 kb bins. 7 clusters were discovered by Louvain clustering in the mouse PFC sample (FIG. 13A). The reads from all the cells in each cluster were then combined to calculate the CG methylation level over neuron type-specific CG-differentially methylated regions (DMRs) previously profiled by snmC-seq. These neuron type-specific CG-DMRs were regions with lower CG methylation level in a specific cell type than all other cell types. In the mouse PFC sample, it was identified that clusters 5 and 7 were excitatory neurons since their CG methylation level in excitatory-neuron-specific CG-DMRs were lower than that in inhibitory-neuron-specific CG-DMRs (FIG. 13B). Clusters 2, 3 and 6 were identified as inhibitory neurons. Clusters 1 and 4 were associated with non-neuronal cells because they showed fairly high methylation rate over most neuron-specific CG-DMRs.

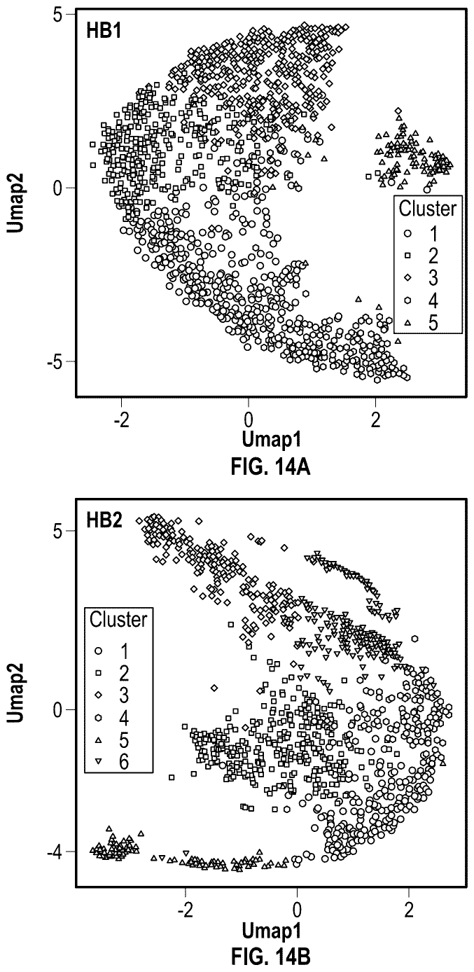

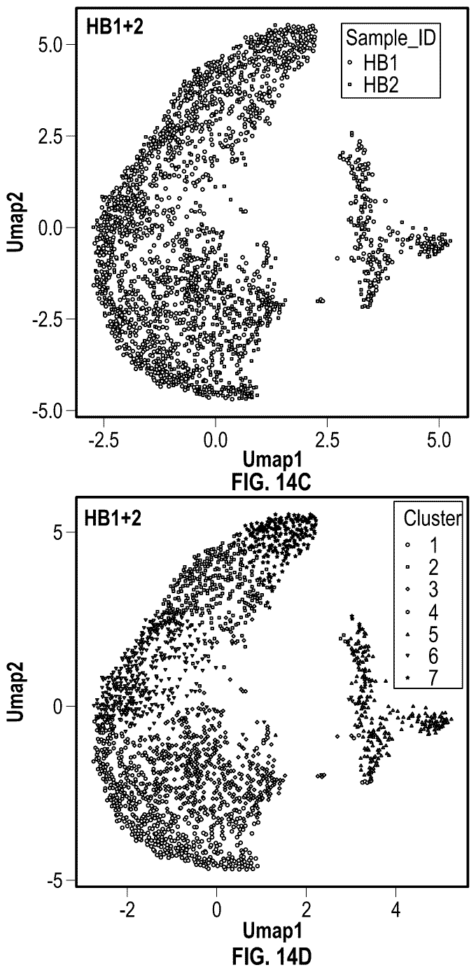

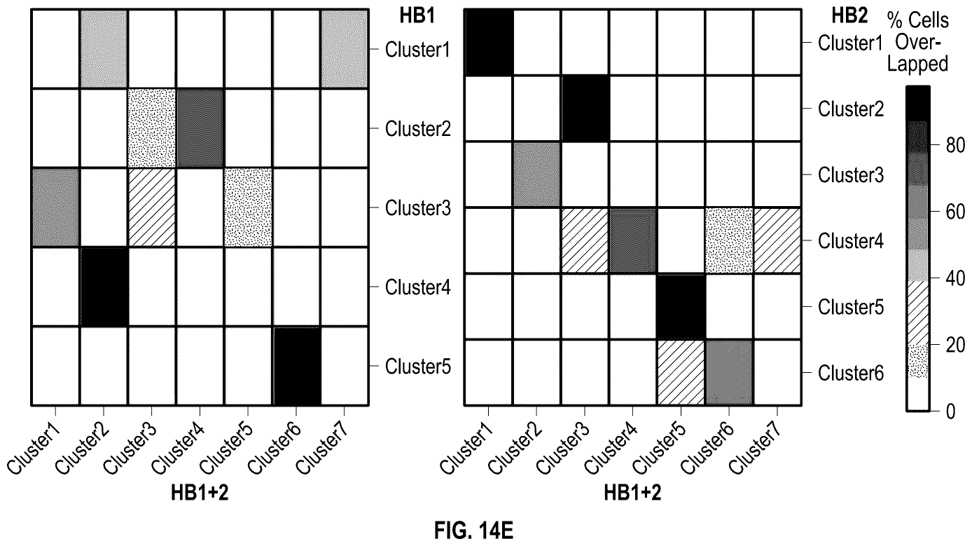

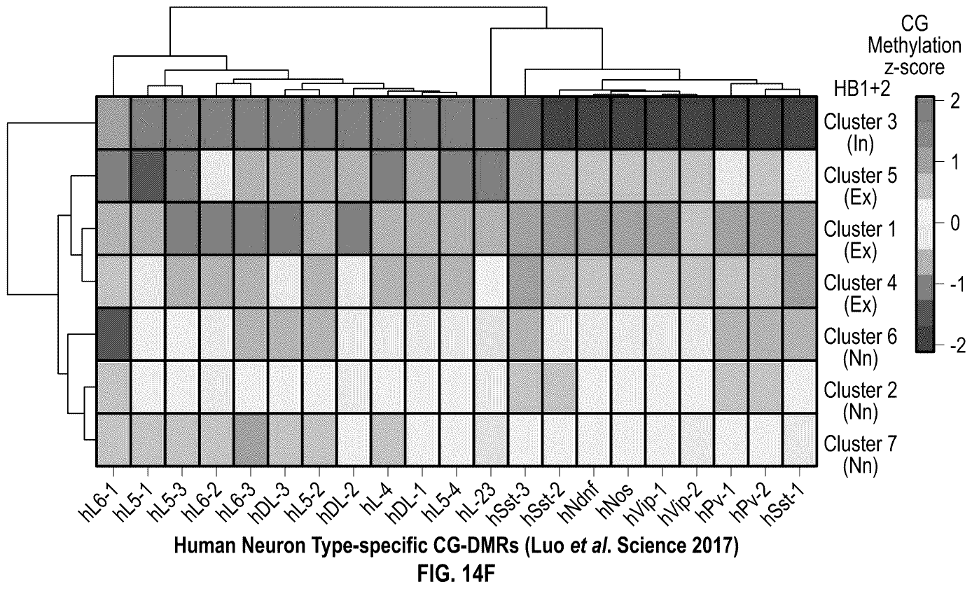

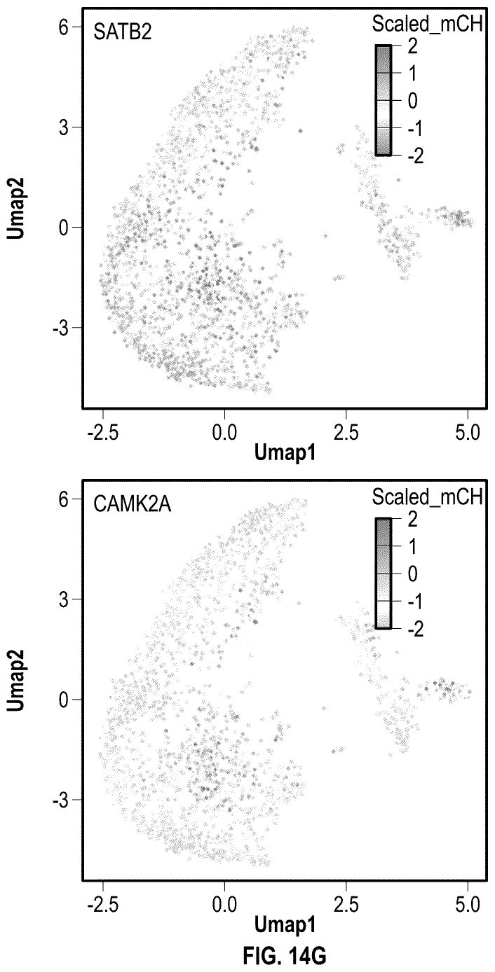

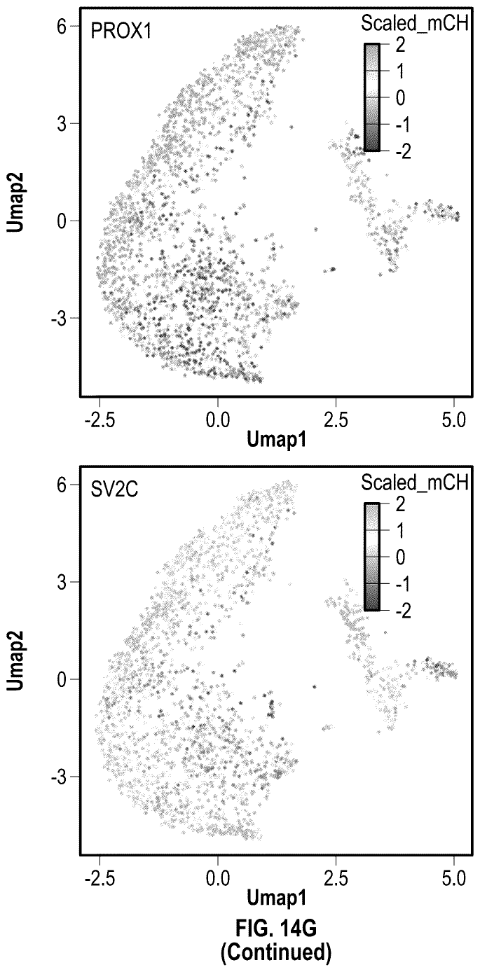

Within this Example, two postmortem human brain samples were further profiled. Human brain 1 (HB1) yielded 1,556 single cells with an average ~41,013 unique reads per cell, while human brain 2 (HB2) yielded 1,257 single cells with an average ~34,630 unique reads per cell. UMAP clustering on the two datasets were first conducted separately. HB1 Drop-BS data yielded 5 clusters (FIG. 14A) and HB2 data yielded 6 clusters (FIG. 14B). Next, the two datasets for co-clustering were combined. The two datasets covered similar UMAP space as shown in FIG. 14C. The combined data with a total of 2,813 single cells yielded 7 clusters (FIG. 14D). It was confirmed that all the clusters yielded by the individual human brain datasets could be mapped to 1-3 clusters among the 7 clusters of the combined dataset (FIG. 14E). 4 out of 5 HB1 clusters and 6 out of 6 HB2 clusters were largely matched to single clusters in the combined dataset (i.e., >53% of single cells in a HB1/2 cluster belonged to a single cluster in the combined data). This demonstrated that the UMAP clustering of the Drop-BS brain data yielded consistent results. Based on CG methylation z-scores over human neuron type-specific CG-DMRs for the combined clusters, cluster 3 was identified to be inhibitory neurons, clusters 1, 4, 5 to be excitatory neurons, and clusters 2, 6, 7 to be non-neuronal cells (FIG. 14F).

The mCH levels were examined at the genome-wide level and in various functional elements for these clusters as shown below in Table 2. Variation in the mCH level across these clusters were observed in both the global average and on functional elements including genic regions, transcription factor binding sites (TFBS), promoter regions, and CpG islands. Generally, genic regions and TFBS have similar mCH level which was lower than the genome-wide average. Promoter regions presented even lower mCH level than those of genic regions and TFBS, while CpG islands had the lowest mCH/CH among the four types of functional elements.

TABLE 2
The average mCH/CH of each cluster in combined human brain
1 + 2 dataset on various functional elements

| | Cluster 1 | Cluster 2 | Cluster 3 | Cluster 4 | Cluster 5 | Cluster 6 | Cluster 7 |
|---|---|---|---|---|---|---|---|
| Genome wide | 0.035 | 0.0284 | 0.0126 | 0.0325 | 0.0368 | 0.0266 | 0.0287 |
| CpG Islands | 0.0082 | 0.0081 | 0.0071 | 0.008 | 0.0078 | 0.0077 | 0.008 |
| Genic Regions | 0.0318 | 0.0261 | 0.0128 | 0.0296 | 0.0332 | 0.0246 | 0.0265 |
| Intergenic | 0.0378 | 0.0304 | 0.0124 | 0.035 | 0.0402 | 0.0285 | 0.0306 |
| Regions | | | | | | | |
| Promoter | 0.0226 | 0.0197 | 0.0104 | 0.0214 | 0.0228 | 0.0179 | 0.0194 |
| Regions | | | | | | | |
| TFBS | 0.0324 | 0.0265 | 0.0124 | 0.0301 | 0.0336 | 0.0247 | 0.0269 |

Pseudobulk differential analysis was conducted between the excitatory neuron clusters (1, 4, and 5) and the inhibitory neuron cluster (3) and discovered 637 DMRs. 594 DMR-associated genes were identified and included known glutamatergic or GABAergic neuron markers, such as SATB2, CAMK2A, PROX1, SV2C, GRIK3, and SOX6. The Drop-BS data also exhibited expected mCH variation among clusters at these marker genes as shown FIG. 14G.

As demonstrated by the Examples of the disclosure, Drop-BS allows for the processing of a large number of cells or nuclei. The scDNA droplet generation of the disclosure was conducted with rate of ~3,000 droplets per second while barcode droplet generation and fusion with scDNA droplets were conducted at a rate of ~132 droplets per second. Under a standard operation of processing about 2,000 cells (involving about 20,000 barcode beads and about 574,000 droplets), the Drop-BS operation on the microfluidic devices was completed within a combined 1.3 hours, with 10 minutes on the droplet generation device to produce scDNA droplets, 36 minutes on the droplet fusion device, and 30 minutes on the bisulfite droplet device. The entire protocol required 2 days to complete. Therefore, these results demonstrate that up to 10,000 cells may be processed by one operator during the same period (2 days) by increasing the running time on the microfluidic devices or running multiple batches of experiments in the case of the droplet fusion.

### Example 4

### Bisulfite Conversion in Droplets

The bisulfite rate conversion in droplets were further analyzed as previously described in Example 1. In particular, a comparison of various parameters and conditions were evaluated with regard to bisulfite conversion rate. The parameters and results of the bisulfite conversion rate can be found below in Table 3A and Table 3B.

TABLE 3A

| | Droplet | Bisulfite | | | Total | Mapped |
|---|---|---|---|---|---|---|
| | Size | Treatment | Fragmentation | Agarose | reads | reads |
| Sample | (μm) | Time (hr) | by MNase | Droplets | (M) | (M) |
| 1 | 70 | 1.5 | No | No | 11.6 | 6.1 |
| 2 | 70 | 3 | No | No | 24.7 | 16.2 |
| 3 | 90 | 3 | No | No | 26.8 | 17.3 |
| 4 | 70 | 4 | No | No | 10.2 | 6.2 |
| 5 | 90 | 4 | No | No | 10.7 | 8.1 |
| 6 | 70 | 1.5 | Yes | No | 14.2 | 11.2 |
| 7 | 70 | 1.5 | Yes | Yes | 7.7 | 6.1 |

TABLE 3B

| | Mapping | Bisulfite |
|---|---|---|
| | Efficiency | Conversion |
| Sample | (%) | Rate (%) |
| 1 | 52.6 | 8.8 |
| 2 | 65.7 | 79.0 |
| 3 | 64.8 | 91.8 |
| 4 | 60.9 | 90.9 |
| 5 | 75.9 | 95.0 |
| 6 | 78.8 | 99.5 |
| 7 | 79.9 | 99.5 |

As can be seen in Tables 3A and 3B, a high bisulfite conversion rate can be reached when the DNA is pre-fragmented by an enzyme. The two samples that had fragmentation by MNase were able to achieve much higher bisulfite conversion rates in comparison to those samples that did not undergo fragmentation.

### Example 5

### Use of Devices within Systems for Single-Cell Sequencing

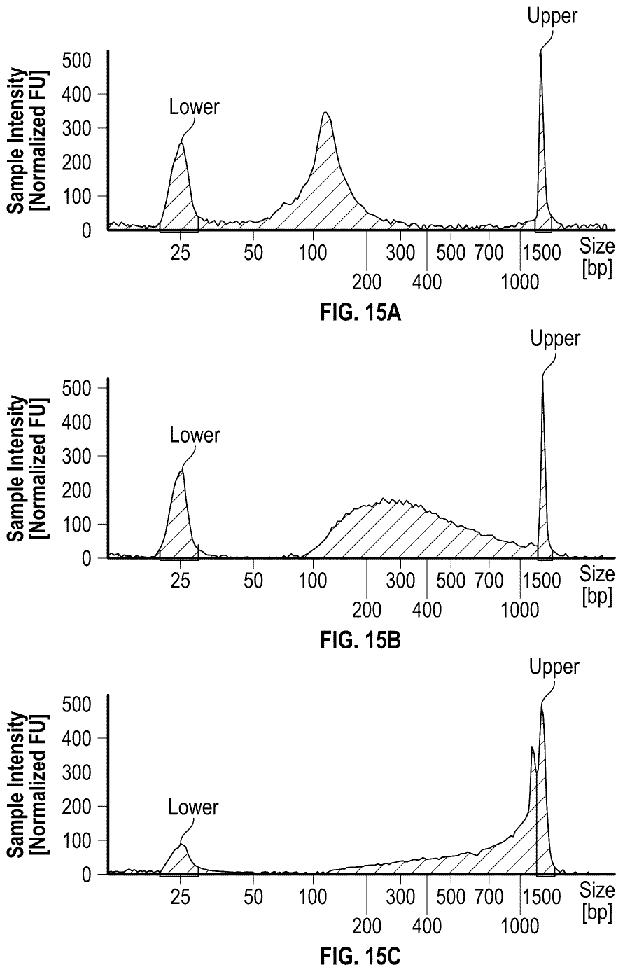

A droplet generation microfluidic device (FIG. 3A) was used to encapsulate single nuclei and lysis buffer containing micrococcal nuclease (MNase) into droplets (~35 μm in the diameter) for single-nucleus lysis and genomic DNA fragmentation (FIG. 2c). The flow rates and concentrations were set so that ~10% of the droplets contained single cells and the probability of having double occupancy in a droplet was low (<0.5%). It was found that the DNA fragment size affected the final library yield substantially. Generally, short DNA fragments (<150 bp) due to over-digestion were removed by SPRIselect bead selection and under-digestion of DNA yielded less input fragments for barcoding. The MNase digestion condition was optimized through CaCl₂ concentration to maximize the DNA output. The results are shown in FIGS. 15A, 15B, and 15C.

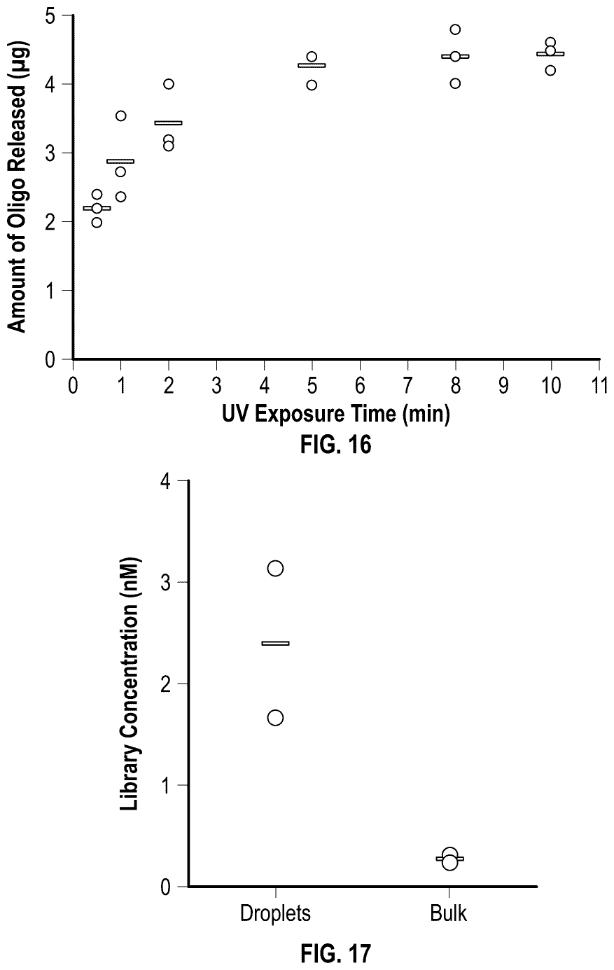

Three inlets of the droplet fusion device (FIG. 3B) were further used to produce droplets containing single barcode beads and end-repair/ligation reagents (at about 7.0% bead occupancy; FIG. 3D) while two side inlets allowed re-injection and spacing of the scDNA droplets generated from step 1 (FIG. 3E). An alternating current (AC) voltage was applied via an adjacent salt channel to produce dielectrophoresis (DEP)-based fusion between the scDNA and single-barcode bead droplets (FIG. 3F). On average, about 80% of the barcode droplets were fused with the scDNA droplets. For the step of scDNA ligation and barcoding, the fused droplets were collected into a tube and exposed to UV (FIG. 16). The barcoded oligonucleotides were released from barcode beads due to breakage of the photocleavable linker and appended to the fragmented gDNA via ligation reaction in droplets, completing scDNA barcoding. The droplets were broken after barcoding.

In conducting the droplet-based bisulfite conversion, it was discovered that conducting bisulfite conversion in droplets substantially increased the library concentration by a factor of 9 compared to bulk conversion in a tube, as shown in FIG. 17. The bisulfite droplet device as shown in FIG. 4 was used to generate droplets (~35 μm in the diameter) containing barcoded DNA and bisulfite. The droplets were incubated for bisulfite conversion to achieve a conversion rate of 99.0% (tested using unmethylated lambda DNA). The droplets were then broken to pool the DNA. Random priming and indexing-PCR-based amplification were then conducted to generate the i5/i7-indexed library for sequencing.

The above specification provides a description of the manufacture and use of the disclosed compositions and methods. Since many embodiments can be made without departing from the spirit and scope of the disclosure, the disclosure resides in the claims.

What is claimed is:

1. A method of single-cell bisulfite sequencing, the method comprising:
generating a plurality of single-cell DNA (scDNA) droplets comprising a single cell or nucleus from a population of cells or nuclei and a lysis buffer comprising an enzyme;
lysing the single cell or nucleus within each of the plurality of scDNA droplets resulting in a plurality of fragmented scDNA within each of the plurality of scDNA droplets;
generating a plurality of barcode droplets each comprising a barcode bead oligonucleotide;
fusing the plurality of scDNA droplets and the plurality of barcode droplets to form a plurality of barcoded scDNA droplets each comprising a barcoded scDNA;
breaking the plurality of barcoded scDNA droplets;
co-encapsulating the barcoded scDNA with a bisulfite reagent to form a plurality of bisulfite droplets;
converting the barcoded scDNA to bisulfite-converted scDNA within the plurality of bisulfite droplets; and
determining methylation profile of the fragmented scDNA.

2. The method of claim 1, wherein the fragmented scDNA has a size ranging from 100 bp to about 1000 bp prior to the step of converting the barcoded scDNA to the bisulfite-converted scDNA.

3. The method of claim 1 or 2, wherein the enzyme comprises micrococcal nuclease.

4. The method of any one of claims 1-3, wherein the plurality of bisulfite droplets each have a diameter of about 20 μm to about 200 μm.

5. The method of any one of claims 1-4, wherein the lysis buffer further comprises CaCl₂ in a concentration range of about 0.1 mM to about 0.3 mM.

6. The method of any one of claims 1-5, wherein the generation step of the plurality of scDNA droplets occurs before and/or after the generation step of the plurality of barcode droplets.

7. The method of any one of claims 1-6, wherein the plurality of scDNA droplets are generated at a rate in the range of about 1000 droplets per second to about 4000 droplets per second, and wherein the plurality of scDNA droplets each have a diameter in the range of about 15 μm to about 50 μm.

8. The method of any one of claims 1-7, wherein each of the barcode bead oligonucleotides comprise the following in order from (i) to (iii):
(i) a bead for carrying barcode oligonucleotides;
(ii) at least one photocleavable linker; and
(iii) at least one barcode oligonucleotide sequence comprising:
a. a PCR priming site comprising the sequence CAAGCAGAAGACGGCATACGAGAT (SEQ ID NO: 2);
b. a cell barcode sequence having a list of 10-20 nucleotides comprising A, G, T, or a combination thereof; and
c. a ligation site sequence having a list of 10-50 nucleotides comprising A, G, C, T, or a combination thereof.

9. The method of claim 8, wherein a complementary oligonucleotide comprising 19 nucleotides is annealed to the ligation site to form a double-stranded tail at a 3′-end of the barcode bead oligonucleotide for ligating to a single fragmented scDNA from the plurality of fragmented scDNA to form the barcoded scDNA within the plurality of barcoded scDNA droplets.

10. The method of claim 8, wherein the plurality of barcoded scDNA droplets are exposed to ultraviolet (UV) radiation to remove the bead from each of the at least one barcode oligonucleotide sequence via breakage of each of the at least one photocleavable linker.

11. The method of any one of claims 1-10, wherein the bisulfite reagent comprises a bisulfite salt selected from the group consisting of sodium bisulfite and ammonium bisulfite.

12. The method of any one of claims 1-11, wherein the bisulfite-converted scDNA further undergoes one or more steps comprising random priming and/or indexing PCR for generating a bisulfite sequenced nucleic acid library.

13. The method of claim 12, wherein the indexing PCR comprises indexing-PCR-based amplification to generate i5- and/or i7-indexed libraries.

14. The method of any one of claims 1-13, wherein the method produces a bisulfite sequenced nucleic acid library of about 1000 to about 10,000 single cells within a period of about 2 days, and provides a bisulfite conversion rate of at least 95%.

15. The method of any one of claims 1-14, wherein the converting of the barcoded scDNA to the bisulfite-converted scDNA within the plurality of bisulfite droplets reduces DNA degradation.

16. The method of any one of claims 1-15, wherein the plurality of scDNA droplets, the plurality of barcode droplets, the plurality of barcoded scDNA droplets, and the plurality of bisulfite droplets are water-in-oil droplets.

17. A system for single-cell sequencing, the system comprising:
a droplet generation device for generating a plurality of scDNA droplets, the plurality of scDNA droplets comprising a single cell or nucleus from a population of cells or nuclei and a lysis buffer comprising an enzyme;
a barcode droplet generation unit within a droplet fusion device, wherein the barcode generation unit generates a plurality of barcode droplets, each comprising a barcode bead oligonucleotide;
the droplet fusion device for fusing the plurality of scDNA droplets and the plurality of barcode droplets to form a plurality of barcoded scDNA droplets each comprising a barcoded scDNA; and
a bisulfite droplet device for co-encapsulating the barcoded scDNA with a bisulfite reagent to form a plurality of bisulfite droplets.

18. The system of claim 17, wherein the droplet generation device comprises three separate inlets each attached to a pump to separately administer the single cell or nucleus from the population of cells or nuclei, the lysis buffer, and an oil to form the plurality of scDNA droplets.

19. The system of claim 17 or 18, wherein the plurality of scDNA droplets are received in a scDNA droplets inlet within the droplet fusion device prior to forming the plurality of barcoded scDNA droplets.

20. The system of any one of claims 17-19, wherein the droplet fusion device further comprises one or more electrode channels comprising a salt solution for conducting dielectrophoresis-based droplet fusion to fuse the plurality of scDNA droplets and the plurality of barcode droplets to form the plurality of barcoded scDNA droplets.


SEQUENCE LISTING (18 sequences)

SEQ ID NO: 1 - DNA, 73 bp, synthetic construct
  source 1..73: mol_type = other DNA
caagcagaagacggcatacgagatdddddddddddddddgtctcgtgggctcggagatgtgtataagagacag

SEQ ID NO: 2 - DNA, 24 bp, synthetic construct
  source 1..24: mol_type = other DNA
caagcagaagacggcatacgagat

SEQ ID NO: 3 - DNA, 34 bp, synthetic construct
  source 1..34: mol_type = other DNA
gtctcgtgggctcggagatgtgtataagagacag

SEQ ID NO: 4 - DNA, 19 bp, synthetic construct
  source 1..19: mol_type = other DNA
ctgtctcttatacacatct

SEQ ID NO: 5 - DNA, 73 bp, synthetic construct
  source 1..73: mol_type = other DNA
  modified_base 1: mod_base = OTHER; note = uracil
  modified_base 5: mod_base = OTHER; note = uracil
  modified_base 12: mod_base = OTHER; note = uracil
  modified_base 15: mod_base = OTHER; note = uracil
  modified_base 19: mod_base = OTHER; note = uracil
  modified_base 42: mod_base = OTHER; note = uracil
  modified_base 44: mod_base = OTHER; note = uracil
  modified_base 50: mod_base = OTHER; note = uracil
  modified_base 52: mod_base = OTHER; note = uracil
  modified_base 71: mod_base = OTHER; note = uracil
taagtagaagatggtatatgagatdddddddddddddddgttttgtgggtttggagatgtgtataagagatag

SEQ ID NO: 6 - DNA, 19 bp, synthetic construct
  source 1..19: mol_type = other DNA
  modified_base 1: mod_base = OTHER; note = uracil
  modified_base 5: mod_base = OTHER; note = uracil
  modified_base 7: mod_base = OTHER; note = uracil
  modified_base 13: mod_base = OTHER; note = uracil
  modified_base 15: mod_base = OTHER; note = uracil
  modified_base 18: mod_base = OTHER; note = uracil
ttgttttttatatatattt

SEQ ID NO: 7 - DNA, 42 bp, synthetic construct
  source 1..42: mol_type = other DNA
tcgtcggcagcgtcagatgtgtataagagacagnnnnnnnnn

SEQ ID NO: 8 - DNA, 33 bp, synthetic construct
  source 1..33: mol_type = other DNA
tcgtcggcagcgtcagatgtgtataagagacag

SEQ ID NO: 9 - DNA, 73 bp, synthetic construct
  source 1..73: mol_type = other DNA
ctatctcttatacacatctccaaacccacaaaacvvvvvvvvvvvvvvvatctcatataccatcttctactta

SEQ ID NO: 10 - DNA, 58 bp, synthetic construct
  source 1..58: mol_type = other DNA
gtctcgtgggctcggagatgtgtataagagacagtaagtagaagatggtatatgagat

SEQ ID NO: 11 - DNA, 107 bp, synthetic construct
  source 1..107: mol_type = other DNA
ctatctcttatacacatctccaaacccacaaaacvvvvvvvvvvvvvvvatctcatataccatcttctacttactgtctcttatacacatctccgagcccacgagac

SEQ ID NO: 12 - DNA, 33 bp, synthetic construct
  source 1..33: mol_type = other DNA
ctgtctcttatacacatctgacgctgccgacga

SEQ ID NO: 13 - DNA, 107 bp, synthetic construct
  source 1..107: mol_type = other DNA
gtctcgtgggctcggagatgtgtataagagacagtaagtagaagatggtatatgagatdddddddddddddddgttttgtgggtttggagatgtgtataagagatag

SEQ ID NO: 14 - DNA, 29 bp, synthetic construct
  source 1..29: mol_type = other DNA
aatgatacggcgaccaccgagatctacac

SEQ ID NO: 15 - DNA, 14 bp, synthetic construct
  source 1..14: mol_type = other DNA
tcgtcggcagcgtc

SEQ ID NO: 16 - DNA, 21 bp, synthetic construct
  source 1..21: mol_type = other DNA
gtctcgtgggctcggagatgt

SEQ ID NO: 17 - DNA, 24 bp, synthetic construct
  source 1..24: mol_type = other DNA
atctcgtatgccgtcttctgcttg

SEQ ID NO: 18 - DNA, 29 bp, synthetic construct
  source 1..29: mol_type = other DNA
gtgtagatctcggtggtcgccgtatcatt